(19) United States
(12) Patent Application Publication
(10) Pub. No.: US 2005/0244830 A1
(43) Pub. Date: Nov. 3, 2005

(54) Quantitative multiplex amplification on a genomic scale, and applications for detecting genomic rearrangements

(76) Inventors: Thiery Frebourg, Rouen (FR); Mario Tosi, Maisons-Alfort (FR); Gregory Raux, Rouen (FR)

Correspondence Address: NIXON & VANDERHYE, PC, 901 NORTH GLEBE ROAD, 11TH FLOOR, ARLINGTON, VA, 22203, US

(21) Appl. No.: 10/521,721

(22) PCT Filed: Jul. 18, 2003
(86) PCT No.: PCT/EP03/08476

§ 371 Date: Apr. 18, 2005

(51) Int. Cl. C12Q 1/68; C07H 21/04

(52) U.S. Cl. 435/6; 536/24.3

(57) ABSTRACT

The present application relates to novel composite primers which make it possible to amplify in multiplex at a quantitative level of precision, and to the application of these composite primers for detecting gnomic rearrangements in general and cryptic chromosomal rearrangements in particular. These composite primers contain a tag the sequence of which is absent from or poorly represented in the genome analyzed, and which exhibits a very low propensity to form stable pairings. The composite primers, which contain them, make it possible to carry out multiplex amplifications with quantitative precision on the scale of a genome such as the human genome.

### TECHNICAL FIELD OF THE INVENTION

The present invention relates, in general, to means applicable on the scale of a genome, such as the human genome, which make it possible to amplify nucleotide targets in multiplex while reaching a quantitative level of precision. These means in particular find applications in the field of the detection of genomic rearrangements.

More particularly, the present invention provides amplification primer tags which make it possible to produce composite primers especially suitable for quantitative multiplex amplification on a genomic scale. The present patent application is therefore directed towards these various products, and also methods and uses employing them.

Notably, the means according to the invention make it possible not only to detect rearrangements located within one or more genes, but also chromosomal rearrangements. In particular, the means according to the invention make it possible to detect heterozygous genomic rearrangements which have led to a loss or a gain of genetic material, i.e. unbalanced genomic rearrangements.

Advantageously, the means according to the invention make it possible to detect cryptic chromosomal rearrangements (i.e. rearrangements which are undetectable by standard karyotyping techniques). The means according to the invention are therefore of great value for diagnosing gene and chromosomal diseases, for establishing genomic rearrangement maps, and for isolating genes involved in a genetic disease.

### TECHNOLOGICAL BACKGROUND

Among genetic diseases, those caused by abnormalities in one or more genes (gene diseases) and those whose cause is at the chromosomal level (chromosomal diseases) are distinguished.

Single-gene diseases, such as the muscular dystrophies or cystic fibrosis, are caused by abnormalities in the sequence of one, or even several, genes: they may, for example, be point mutations, or else duplications or deletions of exons.

As regards chromosomal diseases, they are the result of constitutional abnormalities in the number or in the structure of the chromosomes. Such abnormalities can, for example, occur during meiosis from healthy conceivers, or may already be present in one of the two parents.

Abnormalities in number are characterized by an excess or absence of one or more complete chromosomes in a cell. Thus, when the karyotype exhibits 47 chromosomes, there is generally a trisomy, the most well known being trisomy 21.

Abnormalities in the chromosomal structure result from chromosomal breakages followed by abnormal joinings. Their occurrence is most generally familial, with a relatively high frequency in the general population (2.4 per 1000).

When the structural rearrangement is accompanied by neither loss nor gain of genetic material, it is said to be “balanced”, and most of the time remains silent without any phenotypic expression. This is the case, for example, of “Robertsonian” translocations. In the opposite case, it is termed “unbalanced” and it is then generally expressed in the form of sterility or spontaneous abortions, if it is lethal, and in the form of syndromes combining polymalformations and mental retardation, such as DiGeorge syndrome, when this imbalance is viable. This is the case, for example, of chromosomal rearrangements by deletion or insertion.

Detection of Gene Rearrangements:

To detect the possible presence of a gene rearrangement, such as an exon deletion or duplication, the conventional “Southern” method has often been used, which consists in hybridizing on the genomic DNA, cleaved with restriction enzymes, a nucleotide probe specific for the region affected by the gene rearrangement under consideration.

Because of the complexity and the imprecision of this conventional method, several methods using PCR have been developed. Methods based on multiplex PCR are very suitable for rapidly detecting a possible gene rearrangement. It is possible, for example, to find the description of a multiplex PCR method for detecting exon deletions and duplications in the article Duponchel et al. 2001 (Human Mutation 17:61-70 “Rapid detection by fluorescent multiplex PCR of exons deletions and duplications in the C1 inhibitor gene of hereditary angioedema patients”), and the article Charbonnier et al. 2000 (Cancer Research 60:2760-2763 “Detection of exon deletions and duplications of the mismatch repair genes in hereditary nonpolyposis colorectal cancer families using multiplex polymerase chain reaction of short fluorescent fragments”).

The article Duponchel et al. 2001 (Human Mutation 17:61-70) describes a method based on fluorescent multiplex PCR (direct or indirect labelling) which makes it possible to detect heterozygous exon deletions and duplication within a gene which is small in size, namely the C1 inhibitor gene. According to this method, two different specific tags, each consisting of 16 nucleotides, are added to the 5′ end of the sense primers and of the antisense primers, respectively. These 16-nucleotide tags are presented as forming a rare sequence. These tags are suitable for detecting gene rearrangements, but are not suitable for reliably detecting chromosomal rearrangements such as cryptic chromosomal rearrangements, such that they do not constitute a solution to this particular technical problem.

Also found in the article Charbonnier et al. 2000 (Cancer Research 60:2760-2763) is the description of a multiplex PCR method for detecting exon deletions and duplications within the repair genes MLH1 and MSH2 (hereditary non-polyposis colorectal cancer, or HNPCC syndrome). This multiplex PCR method is based on the targeting of short exon fragments (from 92 to 288 bp), and uses for this purpose primers without a tag or with the dinucleotide “GG” which separates the fluorochrome from the hybridization sequence on the labelled primers. Such primers are not suitable for exploration on the scale of an entire genome, but simply on the scale of the few genes targeted. They do not therefore satisfy the technical problem solved by the invention either.

The use of “universal” tags in the context of a multiplex PCR is also described in patent application WO 99/58721 in the name of the Whitehead Institute for Biomedical Research (Inv. David G. Lang and Eric S. Lander; “Multiplex DNA amplification using chimeric primers”). The multiplex PCR method described in application WO 99/58721 is intended to solve the problem of genotyping by simultaneous amplification of many microsatellites or SNPs (Single Nucleotide Polymorphisms).

The technical solution disclosed in application WO 99/58721 therefore solves the qualitative problem of the simultaneous genotyping of a large number of microsatellites, or of SNPs, but cannot satisfy the problem of carrying out quantitative multiplex PCRs such as those which are necessary for the reliable detection of chromosomal rearrangements, and in particular of cryptic chromosomal rearrangements.

U.S. Pat. No. 6,207,372 in the name of Antony P. Shuber (assignor Genzyme Corp.; “Universal Primer Sequence for multiplex DNA amplification”) describes a multiplex PCR method which uses “universal” primers described as making it possible to homogenize hybridization kinetics. These universal primers comprise a sequence which hybridizes to the target DNA and a tag (“UPS tag”) of 17 to 25 bases. This tag is unrelated to the target DNA and is rich in GC at its 5′ end, and has the property of forming stable hybrids with the sequence which is complementary to it, characterized by a melting temperature of greater than 60° C. The sequence presented as being preferred for use as a universal tag is a 20-mer derived from the M13 mp8 bacteriophage. Such tags are suitable for qualitative applications of multiplex PCR, for example for detecting known mutations by cleavage with restriction enzymes or unknown mutations by SSCP (Single-Strand Conformational Polymorphism), but do not satisfy the problem of quantitative multiplex PCR.

To the applicant's knowledge, none of the multiplex PCR methods described in the prior art as being suitable for detecting specific gene rearrangements therefore makes it possible to obtain a quantitative level of precision, and more particularly a quantitative level of precision on the scale of a genome, such as the human genome. None of these methods is transposable to the detection of chromosomal rearrangements, such as cryptic chromosomal rearrangements.

In order to simultaneously amplify several target sequences, the method disclosed in application WO 99/58721 comprises an amplification reaction which is carried out on the DNA using chimeric primers in the presence of high concentrations of magnesium (2.5 to 7.0 millimolar), and at low elongation temperatures (60-70° C.). This amplification reaction may be accompanied by direct labelling (for example using biotin), or else may be followed by a second amplification reaction which places the labelling on the amplification products from the first reaction (indirect labelling).

The chimeric primers disclosed for the amplification reaction on the DNA each consist of a hybridization segment which recognizes its target on the DNA, and a constant tag which should have a weak tendency to hybridize to the DNA. According to the disclosed method, the sequence of the constant portion is selected from bacteriophage, insect or reptile sequences, when the DNA originates from a mammal (and vice versa), since these species differences would be sufficient to reduce the propensity of the constant fragment to hybridize to the DNA. A pair of constant fragments each consisting of 23 nucleotides is explicitly described therein (referenced under SEQ ID NO:1 and SEQ ID NO:2 in WO 99/58721). These two constant tags are derived from the T7 and T3 bacteriophage promoters, respectively.

However, it can be easily verified, for example by computer analysis of the hybridization of these bacteriophage sequences on the human genome, that the choice of constant tags from the sequences of a species which is distant in evolution does not represent a reliable criterion for decreasing the propensity of these tags to hybridize to the DNA.

Detection of Chromosomal Rearrangements

As regards the detection of chromosomal rearrangements, standard karyotyping techniques are still commonly used for diagnosing such rearrangements. The karyotype has, however, been found to be insufficient for detecting certain chromosomal rearrangements, and in particular for detecting subtelomeric chromosomal rearrangements. Other methods, more suitable for detecting cryptic chromosomal rearrangements (i.e. rearrangements undetectable by standard karyotyping techniques), have therefore been developed.

“All-telomere” FISH (Fluorescence In Situ Hybridization) based on the use of 62 commercially available juxtatelomeric probes is the method currently most widely used (Knight S. J. L. et al. 1999, Lancet 354:1676-1681 “Subtle chromosomal rearrangements in children with unexplained mental retardation”; Knight S. J. L. and Flint J. 2000, J. Med. Genet. 37:401-409 “Perfect endings: a review of subtelomeric probes and their use in clinical diagnosis”). The use of this method routinely is, however, limited by the requirements in terms of sample quality (good mitotic index, and good-quality metaphases), the high cost of the reagents and the time required to interpret the results.

CGH (Comparative Genomic Hybridization) has also been used to detect cryptic chromosomal rearrangements (Breen C. J. et al. 1999, J. Med. Genet. 36:511-517 “Applications of comparative genomic hybridization in constitutional chromosome studies”; Ghaffari S. R. et al. 1998, J. Med. Genet. 35:225-233 “A new strategy for cryptic telomeric translocation screening in patients with idiopathic mental retardation”). However, interpretation thereof remains difficult, in particular at the telomeric level, due to the gradual decrease in the fluorescence towards the ends. In addition, CGH has a resolution of between 5 and 10 megabases, which is therefore less than that of FISH, exhibits poor reproducibility, and requires very expensive material.

The CGH method has been combined more recently with the use of DNA chip technology (Pinkel et al. 1998, Nature Genetics 20, 207-211 “High resolution analysis of DNA copy number variation using comparative genomic hybridization to microarrays”, Snijders A M et al., 2001, Nature Genetics 29, 263-264 “Assembly of Microarrays for genome-wide measurement of DNA copy number”). This method combining CGH and DNA chips is based on the use of a very large number of genomic DNA clones immobilized on glass slides. It is described as making it possible to achieve resolutions of less than 1 Mb.

Studying the segregation of microsatellites within a family makes it possible to indirectly detect cryptic chromosomal rearrangements by demonstrating an abnormal segregation of the parental alleles (allele deletion or duplication in the child). However, this method absolutely requires having the parental DNAs, and is limited by the informativeness of the markers.

More recently, a method called MAPH (Multiple Amplifiable Probe Hybridization) has been used to detect gene deletions and duplications (Armour et al. 2000 Nucleic Acids Research 28(2):605-609 “Measurement of locus copy number by hybridization with amplifiable probes”; application WO 00/53804 in the name of the University of Nottingham “Genetic screening”). MAPH is a method which combines the hybridization of specific probes to the genomic DNA immobilized on a membrane and the detection by PCR of the probes effectively hybridized, thus achieving a quantitative level of precision. The principle of MAPH consists in immobilizing the DNA to be analysed, on a filter, and then in hybridizing it with a mixture of specific probes which each carry, at one of their ends, the same constant sequences. The function of these constant sequences is to make it possible to use only one same pair of primers to detect by PCR the various probes retained by hybridization on the filter.

Another method named MLPA (Multiplex ligation-dependent probe amplification) also makes it possible to detect gene deletions or duplications (Schouten J P, McElgunn C J, Waaijer R, Zwijnenburg D, Dieppvens F, Pals G. 2002. Relative quantification of 40 nucleic acid sequences by multiplex ligation-dependent probe amplification. Nucleic Acids Res. 30:e57). In this method, gene dosage is also achieved by molecular hybridisation using specific probes, as in MAPH. However, in MLPA this molecular hybridisation is carried out in solution and two single-stranded probes are annealed next to each other to each target. These single stranded probes also carry tags that are suitable for PCR amplification using a unique set of primers. The enzyme ligase is then added at the end of the hybrididation step and ligates covalently the single-stranded probes bound to the target. Finally, all hybridised sequences are amplified by PCR, using a unique set of primers which recognize the tags present on the single-stranded probes.

Currently, to the applicant's knowledge, no method therefore exists which solves the problem of detecting chromosomal rearrangements using a multiplex PCR. In fact, to solve this problem, it is necessary to be able to conserve a quantitative level of precision, while at the same time moving to the scale of a chromosomal region, of a chromosome, or even of an entire genome such as the human genome. Now, the multiplicity of the targets which must be targeted for detecting chromosomal rearrangements and the variability in the sequence context in which the various genomic targets may be situated considerably complicate the problem of obtaining amplification kinetics which are homogeneous for the various targets within the same multiplex PCR.

In fact, in order to simultaneously amplify the various targets, the various pairs of primers, the number of which is often greater than ten, are all placed together and at the same time under the same operating conditions (medium composition, temperatures, durations), i.e. under single operating conditions which do not generally correspond to the optimum priming conditions for each one of the pairs of primers. Since the hybridization kinetics for each pair of primers are different from one another, this leads to a non-quantitative representation of the various fragments in the final amplification product, or to the appearance of non-specific amplifications.

In addition, there are many molecular interactions to be controlled. Firstly, molecular interactions can occur at the level of the PCR primers themselves via phenomena of competition between primers, such as phenomena of dimerization within a single primer, or within various combinations of primers. Secondly, interactions can occur at the level of the amplified targets. The nucleotide composition of the amplified targets can in fact engender several types of interaction: the formation of intramolecular secondary structures, and the formation of intermolecular complexes between various amplicons. The overall result then corresponds at best to a qualitative multiplex PCR.

No multiplex PCR method of the prior art therefore made it possible to achieve a quantitative level of precision while at the same time keeping total flexibility with regard to the panel of analysable genomic regions, a necessary condition for applications of such a method on the genomic scale. More particularly, no multiplex amplification has previously been developed for detecting cryptic chromosomal rearrangements.

There remains therefore a need for a multiplex amplification method of the multiplex PCR type which would be easy to implement, which would be applicable on the scale of a genome such as the human genome, while at the same time remaining flexible, and which would make it possible to obtain detection not only of genetic rearrangements, but also and especially of chromosomal rearrangements, and more particularly of cryptic chromosomal rearrangements such as subtelomeric rearrangements, at a quantitative level of precision.

### SUMMARIZED DESCRIPTION OF THE INVENTION

The present application provides a technical solution which does not have the drawbacks of the techniques of the prior art, and which has the advantage of making it possible to quantitatively amplify in multiplex several nucleotide targets, while at the same time being applicable on the scale of a genome, such as the human genome.

The inventors have in fact succeeded in developing composite primers, each made up of a hybridization segment and a tag, which make it possible to achieve this aim. To do this, the inventors chose to and have succeeded in constructing composite primers such that no stable pairing forms between composite primers during the multiplex amplifications.

An aspect of the invention lies in the construction of particular tags suitable for the aim pursued and for the technical choices set by the inventors. The inventors have in fact chosen to and succeeded in producing tags which are sufficiently long to make homogeneous the difference in melting temperatures (Tm) between the various hybridization segments used in multiplex, while at the same time being sufficiently short so that these tags do not form any stable pairing during the multiplex amplification. The tags according to the invention each have a nucleotide sequence:
which is absent or rare in the nucleic acid or in the mixture of nucleic acids to which the multiplex amplification will be applied, and
which is also such that the molecular interactions which they may possibly form during the multiplex amplification are relatively unstable.

Advantageously, the tags according to the invention make it possible to obtain multiplex amplification results of quantitative precision. They are also relatively short compared to the tags used in the prior art, which constitutes a considerable technical advantage since, as a result, the primers which contain them can be easily synthesized in labelled form.

Notably, the inventors have succeeded in constructing tags of this type which are applicable on the scale of an entire genome, such as the human genome.

These nucleotide tags are intended to be added at the 5′ end of the hybridization segment, which is itself also selected so as not to introduce stable interactions between composite primers.

The resulting composite primers will make it possible to readily obtain homogeneous hybridization kinetics for the various targets which are amplified in multiplex.

The invention also provides preferential operating conditions for these composite primers, in order to readily obtain reproducible quantitative amplifications. These preferential operating conditions in particular comprise the use, in the reaction mixture, of an agent which facilitates separation of the DNA strands, such as triethylammonium acetate (TEAA) or dimethyl sulphoxide (DMSO), cf. Example 1.

The composite primers according to the invention are particularly suitable for carrying out an amplification of QMPSF (Quantitative Multiplex PCR of Short Fluorescent fragments) type. According to a preferential embodiment of the invention, a set of short targets (the length of which is between 90 and 500 bp, preferably between 90 and 350 bp, more preferably between 90 and 300 bp) is thus chosen.

In addition to the obtaining of a quantitative level of precision even though more than ten or so targets are amplified in multiplex, the technical solution according to the invention has the advantage of great flexibility for the inclusion of new target regions in a multiplex amplification, and it greatly facilitates the steps of optimization of the operating parameters for amplification (determination of the optimum number of amplification cycles and of the optimum hybridization temperature, determination of the optimum concentrations for the various primers).

In terms of applications, the quantitative multiplex amplification according to the invention, which makes it possible to detect gene rearrangements as well as chromosomal rearrangements, has the particular advantage of making it possible to detect cryptic chromosomal rearrangements (cf. Example 1). It also makes it possible to identify and isolate genes involved in genetic diseases (cf. Example 2)

### DETAILED DESCRIPTION OF THE INVENTION

For any nucleic acid or mixture of nucleic acids from which it is desired to amplify at least one target nucleotide sequence, and in particular several target nucleotide sequences in multiplex and at a quantitative level of precision, the present invention provides:
a plurality of pairs of sense and antisense composite primers specially adapted for this purpose, each of said composite primers comprising a hybridization segment and a nucleotide tag, and methods for producing such a plurality of pairs of composite primers, and also
a pair of nucleotide tags, one of which is suitable for use as a tag in a sense composite primer of such a plurality, and the other of which is suitable for use as a tag in an antisense primer of such a plurality, and methods for producing such a pair of tags.

The present patent application is therefore directed towards not only such pairs of tags and such pluralities of pairs of composite primers, but also, individually as products, any tag chosen from a pair of tags according to the invention, and any pair of composite primers and any composite primer which are selected from a plurality of pairs of composite primers according to the invention.

The present patent application is also directed towards the biotechnological, medical and veterinary applications of these products, in particular in terms of detecting genomic rearrangements, and more particularly chromosomal rearrangements.

More particularly, the present patent application is directed towards each one of the subjects defined in the claims as filed.

According to a first aspect of the invention, the present application is directed towards method for producing a plurality of pairs of sense and antisense composite primers specially adapted to the quantitative multiplex amplification of a plurality of target nucleotide sequences present in a nucleic acid or a mixture of nucleic acids, according to which each one of said sense or antisense composite primers produced consists:
of a hybridization segment, respectively sense or antisense, which pairs with said nucleic acid or mixture of nucleic acids, so as to constitute a sense or antisense primer for one of the target nucleotide sequences of the plurality targeted, and
of a nucleotide tag which is attached to the 5′ end of said hybridization segment, but which does not pair with said nucleic acid or mixture of nucleic acids,
and, optionally, of a non-nucleotide component.

The method for producing a plurality of pairs of primers according to the invention is characterized in that the sense and antisense composite primers of said plurality of pairs produced have respective sequences such that:
a) each sense composite primer has, within said plurality, an antisense composite primer with which it forms a pair of sense and antisense composite primers whose respective hybridization segments constitute, with respect to one another, a pair of sense and antisense primers for one of said target nucleotide sequences, each one of said target nucleotide sequences of the plurality targeted thus having a pair of sense and antisense composite primers which is intended for its amplification,
b) all the sense composite primers contain the same nucleotide tag and all the antisense composite primers contain the same nucleotide tag, the tag of the sense composite primers being different from that of the antisense composite primers,
c) the sequence of the tag of the sense composite primers is absent from said nucleic acid or mixture of nucleic acids, or, at the very least, is only present therein at a frequency at least two times less (preferentially at least ten times less) than that predicted statistically for a random sequence of the same length, and the sequence of the tag of the antisense composite primers is absent from said nucleic acid or mixture of nucleic acids, or, at the very least, is only present therein at a frequency at least two times less (preferentially at least ten times less) than that predicted statistically for a random sequence of the same length,
d) the melting temperature of each composite primer (whether it is a sense or antisense primer) has a value 10 to 15° C. higher (limits inclusive) than that which its hybridization segment would exhibit when naked without tag,
e) each composite primer of said plurality of pairs has a sequence such that no composite primer of said plurality of pairs can form, with itself or with another composite primer of the same plurality, complete or partial base pairing for which the variation in free energy ΔG associated with the formation of this possible pairing would be greater than 14 kcal/mol, said variation in free energy ΔG being calculated using the “Primer Premier” software version 5.0 marketed by PREMIER Biosoft International.

The thus selected plurality of sense and antisense composite primer pairs are then produced by any conventional means available to the skilled person, such as by oligonucleotide synthesis.

The present patent application is thus directed towards any plurality of pairs of sense and antisense composite primers which can be obtained using the method according to the invention. Such a plurality of pairs of sense and antisense composite primers is specially adapted to the quantitative multiplex amplification of a plurality of target nucleotide sequences present in a nucleic acid or a mixture of nucleic acids.

The present patent application is thus directed towards any plurality of pairs of sense and antisense composite primers specially adapted to the quantitative multiplex amplification of a plurality of target nucleotide sequences present in a nucleic acid or a mixture of nucleic acids, each one of said sense or antisense composite primers consisting:
of a hybridization segment, respectively sense or antisense, which pairs with said nucleic acid or mixture of nucleic acids, so as to constitute a sense or antisense primer for one of the target nucleotide sequences of the plurality targeted, and
of a nucleotide tag which is attached to the 5′ end of said hybridization segment, but which does not pair with said nucleic acid or mixture of nucleic acids,
and, optionally, of a non-nucleotide component,
   characterized in that:
a) each sense composite primer has, within said plurality, an antisense composite primer with which it forms a pair of sense and antisense composite primers whose respective hybridization segments constitute, with respect to one another, a pair of sense and antisense primers for one of said target nucleotide sequences, each one of said target nucleotide sequences of the plurality targeted thus having a pair of sense and antisense composite primers which is intended for its amplification,
b) all the sense composite primers contain the same nucleotide tag and all the antisense composite primers contain the same nucleotide tag, the tag of the sense composite primers being different from that of the antisense composite primers,
c) the sequence of the tag of the sense composite primers is absent from said nucleic acid or mixture of nucleic acids, or, at the very least, is only present therein at a frequency at least two times less (preferentially at least ten times less) than that predicted statistically for a random sequence of the same length, and the sequence of the tag of the antisense composite primers is absent from said nucleic acid or mixture of nucleic acids, or, at the very least, is only present therein at a frequency at least two times less (preferentially at least ten times less) than that predicted statistically for a random sequence of the same length,
d) the melting temperature of each composite primer (whether it is a sense or antisense primer) has a value 10 to 15° C. higher (limits inclusive) than that which its hybridization segment would exhibit when without tag,
e) each composite primer of said plurality of pairs has a sequence such that no composite primer of said plurality of pairs can form, with itself or with another composite primer of the same plurality, complete or partial base pairing for which the variation in free energy ΔG associated with the formation of this possible pairing would be greater than 14 kcal/mol, said variation in free energy ΔG being calculated using the “Primer Premier” software version 5.0 marketed by PREMIER Biosoft International.

Step c) reflects the fact that the tags used in the composite primers in accordance with the invention should be absent from or rare in the nucleotide material on which the multiplex amplification will be applied.

Steps b), c), d) and e) reflect the fact that the tags should be sufficiently long to make the melting temperatures of the various hybridization segments homogeneous (increase in the ΔTm of the composite primers), while at the same time remaining sufficiently short so as not to introduce stable interactions between composite primers. This also makes it possible to reinforce the specificity of the resulting composite primers.

It should also be noted that the characteristics of the nucleotide tags according to the invention (in particular their low stability of interaction) differ radically from those which have previously been proposed for tags used in qualitative multiplex PCR, since the length and the composition of the tags proposed for the qualitative multiplex PCR necessarily make them very stable in their pairings.

Step e) reflects the fact that, by virtue of these particular tags, the resulting composite primers do not form any stable pairing within the same plurality. The term “stability” used in the present application in relation to the possible duplex which an oligo-nucleotide such as a tag, a hybridization segment or a composite primer can form, by base pairing, is intended to be understood according to the meaning and the scope given to it by those skilled in the art. It thus has a meaning and a molecular and thermodynamic scope which can be defined by the parameter ΔG of variation in free energy associated with the formation of a possible pairing. The stability of an oligonucleotide duplex is in fact determined according to the geneic formula ΔG=G_{product}−G_{reagents}, which makes it possible to calculate the change in free energy produced by this chemical reaction. The ΔG values, which define the stability of a pairing between nucleotides, are most commonly negative, which indicates that the reactions between oligonucleotides occur spontaneously in the direction “reagents (separate oligonucleotides)→product (partial or total duplex)”, any chemical reaction taking place spontaneously in the direction of a decrease in free energy. It is, however, easier to compare the stabilities of various oligonucleotide pairings by considering only the absolute values for ΔG, a high absolute value |ΔG| expressing a high stability, a low |ΔG| value expressing a low stability.

Unless otherwise indicated, all the ΔG values indicated in the present application were obtained using version 5.0 of the software marketed under the trademark “Primer Premier”. This software is available from the company PREMIER Biosoft International, 3786 Corina Way, Palo Alto, Calif. 94303-4504, USA (http://www.premierbiosoft.com). Software is in fact commercially available which, using the data of two sequences, calculates the variation in free energy ΔG associated with the formation of a possible pairing between these two sequences. The “Primer Premier” software is an example thereof. It follows the method described by Breslauer K J et al. 1986 (Proc. Natl. Acad. Sci. USA, June 1986, Vol. 83, pages 3746-3750, “Predicting DNA duplex stability from the base sequence”) Briefly, the method described by Breslauer K J et al., 1986 uses the following calculation:
ΔG=−(Δgᵢ+Δg_{sym})+ΣₓΔgₓ
with:
Δgᵢ being equal to 5 kcal for the duplexes containing G-C base pairs, and being equal to 6 kcal for the duplexes composed exclusively of A·T base pairs,
Δg_{sym} being equal to 0.4 kcal for the duplexes formed from a self-complementary sequence, and being equal to 0 kcal for the duplexes formed from two complementary sequences,

ΣₓΔgₘ being equal to the sum of the relative stabilities of each interaction, known as “nearest-neighbour interaction”, which can be observed between the two stands of the duplex, according to the following values for Δgₓ:

| Nearest-neighbour | |
|---|---|
| interaction | Δgₓ (in kcal/mol) |
| AA/TT | 1.9 |
| AT/TA | 1.5 |
| TA/AT | 0.9 |
| CA/GT | 1.9 |
| GT/CA | 1.3 |
| CT/GA | 1.6 |
| GA/CT | 1.6 |
| CG/GC | 3.6 |
| GC/CG | 3.1 |
| GG/CC | 3.1 |

The interactions known as “nearest-neighbour interactions” correspond to the interactions which result from the presence, on one strand of the duplex, of a base pair which is complementary to the base pair which corresponds to it on the other strand of the duplex. Thus, for DNA duplexes, ten types of nearest-neighbour interaction are possible: dAA/dTT, dAT/dAT, dCG/dCG, dCT/dAG, dGA/dTC, dGC/dGC, dGG/dCC, dGT/dAC, dTA/dTA and dTG/dCA. For each one of these nearest-neighbour interactions which are identified in the duplex, the relative stability value Δgₓ which corresponds to it is attributed, the sum of all these relative stabilities giving the value ΣₓΔgₓ. This sum of relative stabilities is then weighted by the values for the parameters Δgᵢ and Δg_{sym}, as indicated above.

In the present application, the terms such as “oligo-nucleotide”, “amplification” or “primer” have the usual meanings given to them in the field of molecular biology in general, and of polymerase chain amplification reactions in particular.

Briefly, the term “oligonucleotide” usually implies a chain of more than two nucleotides, preferentially of more than three nucleotides, up to about 30 nucleotides. As regards the oligonucleotide tags according to the invention, a length of between 8 and 18, preferentially between 8 and 14, nucleotides, limits inclusive, has been found to be suitable for application to the human genome.

The term “amplification” refers to the operation by which the number of copies of a target nucleotide sequence present in a sample is multiplied, that is to say, briefly, a process which uses an enzyme with polymerase activity and a primer, or pair of primers, to increase the amount of a particular nucleotide sequence, called target, by polymerization of the four bases dATP, dTTP, dGTP and dCTP, according to the nucleotide chain of the target sequence.

The target nucleotide sequence is generally contained within a nucleic acid or mixture of nucleic acids (nucleotide matrix), from which it is sought to amplify it. In certain cases, however, the target nucleotide sequence and the nucleotide matrix are merely one and the same entity.

In practice, the amplification is generally carried out by a succession of hybridization elongation-denaturation cycles, and the products derived from an amplification cycle are then used as matrix in the following cycle.

The term “primer” or “amplification primer” refers to an oligonucleotide which is capable of acting as a point of initiation of the synthesis so as to synthesize an extension product which comprises the target nucleotide sequence targeted, i.e. an oligonucleotide the sequence of which is such that this oligonucleotide hybridizes to the target which must be amplified, or to the possible nucleic acid or mixture of nucleic acids in which this target nucleotide sequence is contained, at a site such that and with an affinity such that it is possible to elongate it using an enzyme with polymerase activity by complementarity with the sequence to which it is hybridized, the cyclic repetition of such hybridizations-elongations-denaturations leading to the amplification of said target according to exponential kinetics.

The amplification primers should have a sequence which is sufficiently complementary to the nucleic acid or mixture of nucleic acids to allow the enzyme with polymerase activity to exercise its elongation activity. In order for the amplification reaction to be specific for the target towards which it is directed, it is necessary for the primer(s) used to have a sequence which is completely complementary to the sequence of the target. The amplification primers are generally relatively short (of the order of 15 to 30 nucleotides), but may be longer under certain experimental conditions. They are most commonly used in pairs, each member of the same pair then being chosen such that, once hybridized to the nucleic acid or mixture of nucleic acids, they together form the boundaries of the target.

Said nucleic acid or mixture of nucleic acids can be isolated or synthesized DNA, RNA or cDNA. This nucleic acid or mixture of nucleic acids can be provided in purified or pure form, or else in unpurified form, provided that it remains accessible to the hybridization segments which must pair with it. It can therefore just as equally be a biological or microbiological sample which provides access to its nucleotide content such that a primer may hybridize thereto.

Said nucleic acid or mixture of nucleic acids can be single-stranded or double-stranded (if it is double-stranded, a denaturation step will be provided in order to allow hybridization of the segments contained in the composite primers). The invention can be used on any type of nucleic acid or mixture of nucleic acids. In fact, to the inventors' knowledge, a plurality of pairs of composite primers in accordance with the present invention can be produced for any organism or microorganism for which the entire genome or a significant part of the genome has, to date, been sequenced.

Said nucleic acid or mixture of nucleic acids may thus be derived from animal cells, from plant cells, or from any other organism the genome of which is diploid or polyploid.

For example, said nucleic acid or mixture of nucleic acids may thus be derived from a plant such as maize, wheat, rapeseed, tobacco, or any other plant used for transgenesis or in which a variation in the number of copies of certain genes has a considerable phenotypic effect, for example in relation to growth or to resistance to specific conditions.

Said nucleic acid or mixture of nucleic acids may also be derived from a microorganism, such as yeast or fungus.

Said nucleic acid or mixture of nucleic acids may also be derived from an invertebrate animal, such as a worm, insect, arachnid or mollusc, or from a vertebrate animal, such as a fish, reptile, bird or mammal. For example, the mammalian animal may be a rodent (rabbit, mouse, rat, guinea pig, hamster, for example), a bovine (for example a cow), an ovine (a sheep, a goat, for example) or a porcine animal (for example a pig). According to an advantageous embodiment of the invention, said animal is a mammal, and preferentially a human. According to a particularly advantageous embodiment of the invention, the sequence of the tags of the composite primers according to the invention is absent or rare in the human genome.

The sequence of the human genome is the consensus sequence resulting from the connection of all the sequences produced on human genetic material. The general characteristics of this sequence are described in Lander et al., 2001 (Nature 2001, 409:860-921 “Initial sequencing and analysis of the human genome”). This sequence is available online on the site of the National Center of Biological Information (NCBI): http://www.ncbi.nlm.nih-gov/genome/guide/human.

The search for rare sequences in the human genome can be carried out using the computer program “Basic Local Alignment Score Tools” (BLAST) available online on the site: http://www.ncbi.nlm.nih.gov/BLAST/, by following the instructions which are given therein for searching for short sequences.

When the sequence of the available tags is absent from or rare in the genome of the species to which belongs the nucleotide material on which the multiplex amplification will be applied, for example tags whose sequence is rare in or absent from the human genome, it is then possible, using these tags, to produce composite primers in accordance with the invention which can be used for any targets contained in the genome of the species in question. This is particularly advantageous in terms of range of applicability. As will be illustrated below, such tags, which are absent from or rare in the human genome, are provided by the present invention.

Advantageously, said nucleic acid or mixture of nucleic acids is derived from mammalian cells, and in particular from human cells.

Notably, said nucleic acid or mixture of nucleic acids may be total genomic DNA. Those skilled in the art are aware, from the prior art, of many protocols for extracting, from a biological source, nucleotide material in general (DNA or RNA), and genomic DNA in particular (cf. Maniatis et al. “Molecular Cloning: A Laboratory Manual”, Cold Spring Harbor Laboratory Press, New York). The protocols most commonly used for extracting total genomic DNA, and in particular human total genomic DNA, are those based on proteinase K and those using commercially available affinity columns for DNA. Said nucleic acid or mixture of nucleic acids can therefore correspond to the total genomic DNA of the organism or microorganism from which it is derived, and in particular to human total genomic DNA.

Said nucleic acid or mixture of nucleic acids may also be total or fractionated RNA. It will then be converted into complementary DNA prior to amplifying the desired targets according to the invention. The protocols most commonly used for extracting cellular RNA, and in particular human cellular RNA and for copying it into complementary DNA are described e.g. in the aforementioned methods manual (Maniatis et al. “Molecular Cloning: A Laboratory Manual”, Cold Spring Harbor Laboratory Press, New York).

For applications for medical purposes, a tag will therefore preferably be chosen in which the oligonucleotide chain is not represented in the human genome or is only slightly represented therein. For applications for veterinary purposes, a tag will be chosen in which the oligonucleotide chain is not represented in the genome of the animal considered, or is only slightly represented therein.

When the nucleic acid or the mixture of nucleic acids is the human genome, the present invention demonstrates that it is possible to produce tags in accordance with the invention which are only from 8 to 18 nucleotides, preferentially from 8 to 15 nucleotides, more preferentially from 8 to 14 nucleotides, even more preferentially from 8 to 12 nucleotides, very preferentially 10 nucleotides, long. A short tag, the length of which is, for example, between 8 and 14 nucleotides, makes it possible, compared to a longer nucleotide tag, to synthesize composite primers of better quality, in particular when a label of the fluorochrome type must be associated therewith.

According to a preferential embodiment of the invention, the sequence of the tag of the sense composite primers, and also that of the tag of the antisense composite primers, each consist of a chain of 10 nucleotides the GC content of which is between 20 and 60% (limits inclusive), preferentially between 20% and 50% (limits inclusive), more preferentially between 35 and 45% limits inclusive, very preferentially a GC content of 40%.

Very preferentially, the sequence of the tag of the sense composite primers, and also that of the tag of the antisense composite primers, each consist of a chain of 10 nucleotides such that the complete pairing with the chain of 10 nucleotides which constitutes the sequence completely complementary thereto exhibits a free energy of formation ΔG which does not exceed 11 kcal/mol.

By way of illustration, tags of 10 nucleotides specially adapted to analysing the human genome are provided in the present application, namely the tags of sequence CGT TAG ATA G (SEQ ID NO:1) and of sequence GAT AGG GTT A (SEQ ID NO:2), and the sequences complementary to SEQ ID NO:1 and SEQ ID NO:2 (respectively: CTA TCT AAC G, SEQ ID NO:47, and TAA CCC TAT C, SEQ ID NO:48). The sequence SEQ ID NO:1 and the sequence complementary thereto are, advantageously, used as a sense primer tag, and the sequence SEQ ID NO:2 and the sequence complementary thereto as an antisense primer tag.

The present patent application is therefore more particularly directed towards the following pairs of tags:
the sequences SEQ ID NO:1 and SEQ ID NO:2 respectively,
the sequence SEQ ID NO:1 and the sequence complementary to SEQ ID NO:2 (SEQ ID NO:48) respectively,
the sequence complementary to SEQ ID NO:1 (SEQ ID NO:47) and the sequence SEQ ID NO:2 respectively, and
the sequence complementary to SEQ ID NO:1 (SEQ ID NO:47) and the sequence complementary to SEQ ID NO:2 (SEQ ID NO:48) respectively.

The present patent application is thus directed towards any plurality of sense and antisense composite primers, for which the tag of the sense composite primers and/or that of the antisense composite primers is/are selected from the group consisting of the sequence of SEQ ID NO:1, the sequence of SEQ ID NO:2, and the sequences complementary thereto (SEQ ID NO:47 and SEQ ID NO:48, respectively), or else for which the sequence of the tag of the sense composite primers and that of the antisense composite primers form a pair of sequences selected from the group consisting of the following pairs of sequences:
the sequence SEQ ID NO:1 and the sequence of SEQ ID NO:2,
the sequence of SEQ ID NO:1 and the sequence complementary to SEQ ID NO:2 (SEQ ID NO:48),
the sequence complementary to SEQ ID NO:1 (SEQ ID NO:47) and the sequence SEQ ID NO:2,
the sequence complementary to SEQ ID NO:1 (SEQ ID NO:47) and the sequence complementary to SEQ ID NO:2 (SEQ ID NO:48).

The present application also provides several examples of pairs of hybridization segments which, when they are associated with a tag in accordance with the invention, form composite primers according to the invention. Use may thus be made of the sequences of:
SEQ ID NO:3 and SEQ ID NO:4, as sense and antisense hybridization segments, respectively, for a short exon fragment located on the PRODH gene,
SEQ ID NO:7 and SEQ ID NO:8, as sense and antisense hybridization segments, respectively, for a short exon fragment located on the UFD1L gene,
SEQ ID NO:9 and SEQ ID NO:10, as sense and antisense hybridization segments, respectively, for a short exon fragment located on the ARVCF gene,
SEQ ID NO:11 and SEQ ID NO:12, as sense and antisense hybridization segments, respectively, for a short exon fragment located on the HSPOX2 gene,
SEQ ID NO:13 and SEQ ID NO:14, as sense and antisense hybridization segments, respectively, for a short exon fragment located on the HIRA gene,
SEQ ID NO:27 and SEQ ID NO:28, as sense and antisense hybridization segments, respectively, for another short exon fragment located on the PRODH gene,
SEQ ID NO:29 and SEQ ID NO:30, as sense and antisense hybridization segments, respectively, for a short exon fragment located on the USP18 gene,
SEQ ID NO:31 and SEQ ID NO:32, as sense and antisense hybridization segments, respectively, for a short exon fragment located on the DGSA gene,
SEQ ID NO:33 and SEQ ID NO:34, as sense and antisense hybridization segments, respectively, for a short exon fragment located on the DGRC6 gene,
SEQ ID NO:35 and SEQ ID NO:36, as sense and antisense hybridization segments, respectively, for a short exon fragment located on the DGCR2 gene.

By coupling the tag of SEQ ID NO:1 to each sense hybridization fragment, and the tag of SEQ ID NO:2 to each antisense hybridization segment, the sense and antisense composite primers of following respective sequences are obtained:
SEQ ID NO:15 and SEQ ID NO:16 for amplifying a short fragment on the PRODH gene,
SEQ ID NO:19 and SEQ ID NO:20 for amplifying a short fragment on the UFD1L gene,
SEQ ID NO:21 and SEQ ID NO:22 for amplifying a short fragment on the ARVCF gene,
SEQ ID NO:23 and SEQ ID NO:24 for amplifying a short fragment on the HSPOX2 gene,
SEQ ID NO:25 and SEQ ID NO:26 for amplifying a short fragment on the HIRA gene,
SEQ ID NO:37 and SEQ ID NO:38 for amplifying another short fragment on the PRODH gene,
SEQ ID NO:39 and SEQ ID NO:40 for amplifying a short fragment on the USP18 gene,
SEQ ID NO:41 and SEQ ID NO:42 for amplifying a short fragment on the DGSA gene,
SEQ ID NO:43 and SEQ ID NO:44 for amplifying a short fragment on the DGRC6 gene,
SEQ ID NO:45 and SEQ ID NO:46 for amplifying a short fragment on the DGCR2 gene.

These composite primers are of use for exploring human chromosomal region 22q11. As illustrated in examples 1 and 2 below, the pairs of sense and antisense composite primers (SEQ ID NO:15; SEQ ID NO:16), (SEQ ID NO:19; SEQ ID NO:20), (SEQ ID NO:21; SEQ ID NO:22) and (SEQ ID NO:23; SEQ ID NO:24) have allowed a quantitative multiplex amplification of their targets, from total human genomic DNA. This plurality of pairs of composite primers according to the invention has thus made it possible to determine the boundaries of the deletion which is observed in chromosomal region 22q11 in the context of DiGeorge syndrome. As illustrated in Example 2 below, the pairs of sense and antisense composite primers (SEQ ID NO:37; SEQ ID NO:38), (SEQ ID NO:39; SEQ ID NO:40), (SEQ ID NO:41; SEQ ID NO:42), (SEQ ID NO:43; SEQ ID NO:44) and (SEQ ID NO:45; SEQ ID NO:46), used in multiplex on human total genomic DNA, have made it possible to focus on a particular region within that which is deleted in the context of DiGeorge syndrome and have thus made it possible to identify the PRODH gene as an excellent candidate for involvement in schizophrenia.

A non-rearranged gene can be amplified in multiplex as a control or a standard. For example, when a particular chromosome, such as chromosome 22, is targeted, a target on another chromosome, for example a target on chromosome 2, can be chosen, such as a short exon fragment located in the MSH2 gene (for example, using the sense hybridization segment of SEQ ID NO:5 and the antisense hybridization segment=SEQ ID NO:6, which can be respectively coupled to the tags of SEQ ID NO:1 and SEQ ID NO:2, thus forming the sense and antisense composite primers of respective sequences SEQ ID NO:17 and SEQ ID NO:18).

Composite primers according to the invention can therefore for example comprise, as a hybridization segment associated with the tag of SEQ ID NO:1 or a sequence complementary to SEQ ID NO:1 (SEQ ID NO:47), a sequence selected from the group consisting of the sequences of SEQ ID NO:3, SEQ ID NO:5, SEQ ID NO:7, SEQ ID NO:9, SEQ ID NO:11, SEQ ID NO:13, SEQ ID NO:27, SEQ ID NO:29, SEQ ID NO:31, SEQ ID NO:33 and SEQ ID NO:35 (group of the sense composite primers of Examples 1 and 2 presented below). The composite primers which comprise the sequence of SEQ ID NO:2 or the sequence complementary to SEQ ID NO:2 (SEQ ID NO:48) as a-tag can for example comprise, as an amplification primer, a sequence selected from the group consisting of the sequences of SEQ ID NO:4, SEQ ID NO:6, SEQ ID NO:8, SEQ ID NO:10, SEQ ID NO:12, SEQ ID NO:14, SEQ ID NO:28, SEQ ID NO:30, SEQ ID NO:32, SEQ ID NO:34 and SEQ ID NO:36 (group of the antisense composite primers of Examples 1 and 2 presented below).

Advantageously, the composite primers of the same plurality of pairs each have a hybridization segment the melting temperature Tm of which is between 50 and 65° C., preferentially between 58 and 62° C., all limits inclusive. Very preferentially, the composite primers of the same plurality of pairs each have a melting temperature Tm of greater than 65° C., preferentially of between 68° C. and 72° C., all limits inclusive.

The tags according to the invention can be used without labelling, by using other means, known to those skilled in the art, capable of revealing and measuring PCR products (for example, analysis by mass spectrography of the MALDI-TOF type).

However, to facilitate the step of quantitative measurement of the amplified products, the tags according to the invention can also carry one or more non-oligonucleotide compounds, such as a label which allows quantitative detection of nucleotide products, for example a chemiluminescent compound, a radioactive compound, a fluorophore or a biotin. Preferably, this label is a fluoroscein such as 6-FAM (marketed for example by the company Applied-Biosystems).

A plurality of pairs of composite primers in accordance with the invention comprises at least two pairs of composite primers, and there is no universal upper limit which would be applicable to the number of pairs which can be part of the same plurality while at the same time remaining quantitative. The inventors have, however, been able to note that, by virtue of the present invention, it is possible to simultaneously amplify more than ten targets, for example from 2 to 15 target nucleotide sequences in multiplex from the same starting nucleic acid or mixture of nucleic acids (for example from the total genomic DNA of a human), while at the same time remaining quantitative.

To produce a plurality of pairs of sense and antisense composite primers according to the invention, it is possible to follow a method which comprises the following steps:
a) selected from:
pairs of sense and antisense hybridization segments which each form a pair of sense and antisense primers for one of said target nucleotide sequences, and
nucleotide tags which are absent from said nucleic acid or mixture of nucleic acids, or which at the very least are only present therein at a frequency at least two times less than that predicted statistically for a random sequence of the same length,
   are a plurality of pairs of sense and antisense hybridization segments which covers the plurality of target nucleotide sequences targeted, and
   a pair of nucleotide tags,
   the respective sequences of which are such that:
   when one of the two selected tags is attached to the 0.5° end of each selected sense hybridization segment, and the other of the two selected tags is attached to the 5′ end of each selected antisense hybridization segment, then:
each resulting sense or antisense composite primer has a melting temperature Tm with a value 10 to 15° C. greater (limits inclusive) than that which its hybridization segment would exhibit when naked without tag, and
each resulting sense or antisense composite primer has a sequence such that it cannot form, with itself or with another resulting composite primer, a complete or partial base pairing for which the variation in free energy ΔG associated with the formation of this pairing would be greater than 14 kcal/mol,
b) the plurality of pairs of sense and antisense composite primers which results from the selection of the plurality of pairs of hybridization segments and of the pair of tags made in step a), and of the addition of the sequence of one of the two selected tags to the 5′ end of each sense hybridization segment of the selected plurality, and of the addition of the sequence of the other of the two selected tags to the 5′ end of each antisense hybridization segment of the selected plurality, is produced.

To produce a plurality of pairs of composite primers according to the invention, a method which comprises the following steps can also be followed:
a) a plurality of pairs of sense and antisense hybridization segments is selected:
in which each pair of segments constitutes a pair of sense and antisense primers for each one of the target nucleotide sequences targeted, and
in which no segment can form, with itself or with another segment of this plurality, a complete or partial base pairing for which the variation in free energy ΔG associated with the formation of this possible pairing would be greater than 14 kcal/mol, preferentially 13 kcal/mol, more preferentially 12 kcal/mol,
b) two nucleotide tags are selected:
the respective sequences of which are absent from said nucleic acid or mixture of nucleic acids, or at the very least which are only present therein at a frequency at least two times less than that predicted statistically for a random sequence of the same length, and
which have respective sequences such that their addition, for one, to the 5′ end of each one of the sense hybridization segments selected in step a) and, for the other, to the 5′ end of each one of the antisense hybridization segments selected in step a), does not produce a set of sense and antisense composite primers within which a composite primer would be capable of forming, with itself or with another composite primer of this set, a complete or partial base pairing, the formation of which would correspond to a variation in free energy ΔG of greater than 14 kcal/mol,
c) a plurality of pairs of sense and antisense composite primers is produced by adding the sequence of one of the two tags selected in step b) to the 5′ end of each sense hybridization segment selected in step a), and by adding the sequence of the other of the two tags selected in step b) to the 5′ end of each antisense hybridization segment selected in step a), which constitutes a plurality of pairs of composite primers according to the invention.

Preferentially, said hybridization segments, whether they are sense or antisense, each have (in the absence of tags) a melting temperature Tm of between 50 and 65° C. (limits inclusive), preferably of between 58 and 65° C. (limits inclusive).

The present patent application is thus directed towards any plurality of pairs of composite primers which can be obtained using a method for producing a plurality of pairs of composite primers according to the invention.

The present patent application is also directed towards, individually as a product, any pair of sense and antisense composite primers which is selected from such a plurality.

To the applicant's knowledge, no tag of the prior art has been constructed such that it limits the formation of stable intra- and intermolecular pairing during multiplex PCR.

Now, the novel tags according to the invention and also the composite primers which contain them are precisely chosen so as to avoid the formation of such interactions, and it is here demonstrated that such tags, when they are used under the new operation conditions defined for this invention, make it possible to obtain a quantitative level of precision during the simultaneous amplification of a large number of nucleotide targets, the quantitative performances remaining optimal even above ten simultaneously amplified targets.

A convenient and effective way to select tags which will not significantly increase the ΔG of the segments selected for the production of composite primers in accordance with the invention, and which it will also be possible to use along with a very wide range of hybridization segments, is to follow the method for producing pairs of “universal” tags which follows. This method for producing a pair of “universal” tags comprises the following steps:
a) at least 30 pairs of sense and antisense hybridization segments are chosen:
which each form a pair of sense and antisense primers for a nucleotide target, so as to target at least 30 different nucleotide targets on said nucleic acid or mixture of nucleic acids, and taking care that these at least 30 targets exhibit a uniform distribution throughout the length of said nucleic acid or mixture of nucleic acids, or at the very least in the region(s) in which are found the target nucleotide sequences whose amplification in multiplex is desired, and
each segment of which has a melting temperature Tm of between 50 and 65° C. (limits inclusive), thus constituting a set of pairs of test sense and antisense segments,
b) for each pair of test segments of the set, the maximum value of the variation in free energy ΔG that this pair can exhibit, by partial or complete base pairing of each one of the two segments with itself or with the other segment of the same pair, is determined,
c) two tags of different sequences are selected:
which are not present in said nucleic acid or mixture of nucleic acids, or at the very least which are only present therein at a frequency at least two times less than that predicted statistically for a random sequence of the same length, and
the addition of which, for one, to the 5′ end of each test sense segment and, for the other, to the 5′ end of each test antisense segment, leads to an increase of a value of between 10 and 15° C. (limits inclusive) in the melting temperature Tm of each one of the test segments, and
the addition of which, for one, to the 5′ end of each test sense segment and, for the other, to the 5′ end of each test antisense segment, does not for any of the pairs of test sense and antisense segments lead to an increase of more than 3 kcal/mol in said maximum value ΔG determined for each test pair in step b),
d) the two selected tags are produced.

The set of at least 30 nucleotide targets should be chosen so as to be representative of the region(s) in which are found the target nucleotide sequences whose amplification in multiplex is desired. A set of at least 30 nucleotide targets whose distribution is uniform, in the statistical sense of the term, is therefore chosen. There is no upper limit of number of test nucleotide targets; however, a number of between 30 and 60 is generally sufficient.

Preferentially, this set of at least 30 nucleotide targets is representative of the nucleic acid or mixture of nucleic acids on which the multiplex amplification will be carried out. In this case, a pair of tags is in fact obtained which is in accordance with the present invention and which also constitutes a pair of universal tags for the organism or the microorganism from which the nucleic acid or mixture of nucleic acids is derived. Such a pair of tags has, for example, been obtained for the human species (SEQ ID NO:1 and SEQ ID NO:2, and also the sequences complementary thereto, SEQ ID NO:47 and SEQ ID NO:48). These tags can therefore, in accordance with the present invention, be combined with any segments whose target is on the human genome: the resulting composite primers will always exhibit the quality of allowing a quantitative multiplex amplification.

The present patent application is thus directed towards any pair of nucleotide tags which can be obtained using this method.

When such a pair of tags is available, they can then be added (chemically or virtually) to hybridization segments selected in accordance with the invention, i.e. which do not form, with themselves or with one another, any stable pairing (no ΔG greater than 14 kcal/mol), by adding one of the two tags to the 5′ end of each sense hybridization segment of the plurality of pairs which is intended to be used in a multiplex, and the other of the two tags to the 5′ end of each antisense hybridization segment of this plurality. The resulting sense and antisense composite primers, in virtually all cases, will not form with one another any complete or partial base pairing for which the ΔG would be greater than 14 kcal/mol. Moreover, most of them will form no complete or partial base pairing for which the ΔG would be greater than 12 kcal/mol. If, however, by exception, the coupling of a segment to one of the two selected tags was to result in the maximum threshold of 14 kcal/mol being exceeded, then it would be sufficient to discard the hybridization segment in question, in order to choose another one which has the same functions as the one discarded, but which does not lead to said threshold of 14 kcal/mol being exceeded after coupling with the tag intended for it.

Pairs of tags according to the invention, which exhibit the characteristic of “universality” for the human species, are in particular illustrated by the pairs of tags selected from the group of pairs of tags of respective sequences:
the sequences SEQ ID NO:1 and SEQ ID NO:2,
the sequence SEQ ID NO:1 and the sequence complementary to SEQ ID NO:2 (SEQ ID NO:48),
the sequence complementary to SEQ ID NO:1 (SEQ ID NO:47) and the sequence SEQ ID NO:2,
the sequence complementary to SEQ ID NO:1 (SEQ ID NO:47) and the sequence complementary to SEQ ID NO:2 (SEQ ID NO:48).

The present patent application is also directed towards, individually as a product, any nucleotide tag which is selected from such a pair of tags.

Such a tag according to the invention can commonly consist of 8 to 18 nucleotides, preferably of 8 to 15 nucleotides, more preferentially 8 to 14 nucleotides, even more preferentially 9 to 12 nucleotides, very preferentially 10 nucleotides.

Thus, more particularly targeted are the “universal” tags which are absent from the human genome, or at the very least which are only present therein at a frequency at least two times less (preferentially at least ten times) than that predicted statistically for a random sequence of the same length.

The inventors have in fact been able to develop tags of this length which exhibit the property of “universality” for the human genome, in the sense that they have been constructed relative to the consensus sequence of the genome of the human species (human genome available on the abovementioned NCBI site), and that they are, as a result, suitable for the quantitative multiplex amplification of any target contained in a human genomic DNA. The present patent application is more particularly directed towards any tag the sequence of which is SEQ ID NO:1 or SEQ ID NO:2, or the sequence (of 10 nucleotides) complementary to SEQ ID NO:1 (SEQ ID NO:47) or the sequence (of 10 nucleotides) complementary to SEQ ID NO:2 (SEQ ID NO:48).

The present invention also provides a set of tags which are suitable for use as a tag in the sense composite primers or in the antisense composite primers of a plurality according to the invention. These tags:
each consist of 10 nucleotides,
each have a GC content of between 20% and 60% (limits inclusive), preferentially between 20% and 50% (limits inclusive), more preferentially between 35 and 45%, very preferentially a GC content of 40%, and
are absent from said nucleic acid or mixture of nucleic acids, or which are at the very most only present therein at a frequency at least two times less (preferentially at least ten times less) than that statistically predicted for a random sequence of the same length.

The tags the sequence of which is such that the complete pairing with the chain of 10 nucleotides which constitutes the sequence completely complementary thereto exhibits a free energy of formation ΔG which does not exceed 11 kcal/mol will be preferred. The tags of sequence SEQ ID NO:1, SEQ ID NO:2, sequence complementary to SEQ ID NO:1 (SEQ ID NO:47) or sequence complementary to SEQ ID NO:2 (SEQ ID NO:48) constitute examples of such tags.

A preferential sense or antisense composite primer according to the invention has a “universal” tag according to the invention as tag. More particularly, the present application is directed towards any composite primer the sequence of whose tag is a sequence selected from the group consisting of the sequence of SEQ ID NO:1 and the sequence complementary to SEQ ID NO:1 (these tags can, for example, be used for all the sense composite primers), and any composite primer the sequence of whose tag is a sequence selected from the group consisting of the sequence of SEQ ID NO:2 and the sequence complementary to SEQ ID NO:2 (these tags can, for example, be used for all the antisense composite primers).

The tags of primers according to the invention make it possible to have composite primers which are effective in terms of quantitative precision while at the same time remaining completely evolutive. The tags according to the invention can thus be combined with any desired hybridization segment, and can be used in many biological applications, and in particular for detecting genomic rearrangements, for determining the limits of a genomic rearrangement, and for identifying a gene involved in a genetic disease.

The present patent application is therefore, in general, directed towards any method for amplifying at least one target nucleotide sequence present in a nucleic acid or a mixture of nucleic acids, by hybridizations and elongations of at least one pair of amplification primers, characterized in that said at least one pair of primers is chosen from a plurality of pairs of sense and antisense composite primers according to the invention.

Preferentially, when a quantitative level of precision is effectively desired during a multiplex amplification, the sense and antisense composite primers according to the invention contain sense and antisense hybidization segments which have melting temperatures (Tm) which differ by less than 5° C. For example, the sense and antisense hybridization segments respectively contained in a pair of sense and antisense composite primers according to the invention can each have a Tm of between 50 and 65° C. Once combined with a tag according to the invention, such as SEQ ID NO:1, SEQ ID NO:2 or the sequences complementary thereto (SEQ ID NO:47 and SEQ ID NO:48), the resulting sense and antisense composite primers will then generally each have a Tm of greater than 65° C., and preferably of between 68° C. and 72° C., all limits inclusive.

The method of amplification according to the invention can be applied to any nucleic acid or mixture of nucleic acids in which said target nucleotide sequence(s) is (are) contained. It may, for example, be a nucleic acid or a mixture of nucleic acids derived from human cells or fluids, but equally from non-human (non-human mammals in particular) animal cells or fluids, just as from cells or fluids of plant origin. For diagnostic applications, in particular in the context of the detection of genomic rearrangements, this nucleic acid or mixture of nucleic acids is most commonly of animal, and in particular human, origin. For genetic identification applications, this nucleic acid or mixture of nucleic acids is most commonly of animal, human, microbiological or plant origin. Examples of samples of cells comprise in particular, in the field of chemical applications, samples of blood cells, of epithelial cells and of foetal cells, and of biopsies. Examples of fluid of animal origin comprise blood, urine, cerebrospinal fluid and, in general, any fluid that a healthy or sick organism is liable to exude or contain.

Depending on the primers chosen and on the nucleic acid or mixture of nucleic acids to which the amplification must be applied, those skilled in the art will be able to adjust the suitable operating conditions. If it is chosen to carry out the amplification using the polymerase (i.e. by PCR), conditions for implementation comprise for example:
an amplification medium comprising, in addition to the primers:
from 1.5 to 5.0 mM MgCl₂,
from 10 to 100 mM KCl or (NH₄)₂SO₄ or NaCl
from 10 to 100 mM Tris-HCl, pH 7.5 to 9.0,
100 μM to 500 μM of the four deoxynucleotide triphosphates (dNTPs),
from 25 to 100 units/ml of Taq polymerase (for example, “Thermoprime Plus DNA Polymerase” from ABgene®),
temperatures cycles making it possible to alternate hybridization and elongation and denaturation periods, typically a denaturation period at 94° C. of approximately 5 min, 18 to 27 PCR cycles carried out according to the characteristics below, followed by an elongation step of 5 min at 72° C.:
10 to 30 seconds at 94° C. (denaturations),
15 to 45 seconds at a temperature of between 50 and 60° C. (hybridizations),
20 to 60 seconds at 72° C. (elongations).

Those skilled in the art will also be able, using conventional techniques, to identify and quantify the products of amplification of the various fragments amplified by the method according to the invention. By way of indication, to identify and quantify the products of amplification of the various fragments of a quantitative multiplex PCR, conventional conditions used are, for example:
labelling one of the composite primers of each pair of primers on its 5′ end with a fluorophore,
separating the products of the multiplex PCR by electrophoresis in a DNA sequencing device used in fragment analysis mode,
recording the quantitative profile of the distribution of the fluorescence subsequent to the electrophoresis (called electropherogram),
superposing by computer each one of the electropherograms corresponding to the samples analysed, onto electropherograms corresponding to normal controls.

Any means known to those skilled in the art for identifying and quantitatively analyzing the products of the multiplex PCR can be used in the implementation of the present invention. By way of alternative illustration, the quantitative revelation of the amplification products of the multiplex PCR can also be carried out by a step consisting of hybridization of all of the products of the multiplex PCR on one or more DNA chips (or equivalent membranes) containing sequences specific for each target. In this method of revelation, each one of the fluorescent strands derived from the amplification of one of the targets will be quantitatively attached at a precise position, thus allowing quantitative reading of the amplification of the corresponding fragment, without involving separation by electrophoresis.

According to a particularly advantageous aspect of the invention, the method of amplification can be multiplex and remain quantitative. The present patent application is thus directed towards, according to a preferred embodiment, any method for simultaneously amplifying a plurality of target nucleotide sequences present in a nucleic acid or a mixture of nucleic acids, by hybridizations and elongations of a plurality of pairs of amplification primers, characterized in that said plurality of pairs of amplification primers is a plurality according to the invention.

As indicated above, the inventors have noted that, using human total genomic DNA, it is possible, by virtue of the present invention, to simultaneously amplify more than ten targets, for example from 2 to 15 target nucleotide sequences in multiplex while at the same time remaining quantitative. The upper limit of the number of targets which can be simultaneously analyzed in the same multiplex PCR depends especially on the methods and on the means used to distinguish said targets after amplification and on the precision and the finesse of the computer predictions for the interactions between the primers.

The composite primers according to the invention are particularly suitable for carrying out an amplification of the QMPSF (Quantitative Multiplex PCR of Short Fluorescent fragments) type. Preferentially, said target nucleotide sequences will, for a quantitative multiplex amplification, be chosen to be short fragments, for example from 90 to 500 bp, preferentially from 90 to 350 bp, very preferentially from 90 to 300 bp.

Advantageously, said hybridizations and/or said elongations will, moreover, be carried out in the presence of agents which facilitate DNA strand separation, such as dimethyl sulphoxide (DMSO), triethylammonium acetate (TEAA), or any equivalent agent (cf. Varadaraj K and Skinner DM, 1994, GENE 140: 1-5 “Denaturants or cosolvents improve the specificity of PCR amplification of a G+G-rich DNA using genetically engineered DNA polymerases”, and Baskaran N, Kandpal R P, Bhargava A K, Glynn M W, Bale A, Weissman S M, 1996, Genome Methods 6:633-638 “Uniform amplification of a mixture of nucleic acids with varying GC content”).

Specifically, this preferential embodiment (composite primers according to the invention+DMSO, TEAA or other equivalent compound) has the advantage of further reducing the interactions during the progression of the PCR, whether at the level of the composite primers or at the level of the amplified segments.

In order to obtain a quantitative level of precision, it is also necessary to limit the number of hybridization-elongation-denaturation cycles so as to keep exponential amplification kinetics for each one of the simultaneously amplified targets. Preferentially, said hybridizations-elongations-denaturations will therefore be carried out with successive cycles until the amplification of said at least one target nucleotide sequence is obtained or, where appropriate, the amplifications of said target nucleotide sequences have exponential phase kinetics.

With the tags according to the invention, and using 10% DMSO, the optimum number of cycles is generally between 18 and 27 cycles, for example from 22 to 24 successive cycles of denaturations-hybridizations-elongations, depending on the initial amount of nucleotide material and the particular amplification conditions (cf. example 1 below). If hybridization primers with close melting temperatures have been chosen, the temperature range to be tested to determine the optimum hybridization temperature is, moreover, limited.

According to another particularly advantageous aspect of the invention, all the composite primers and/or pairs of composite primers can be used in equimolar concentration. The choice of appropriate hydridization temperature and of appropriate number of cycles is therefore simplified.

In the end, by virtue of the tags according to the invention, those skilled in the art will only have a small number of combinations [number of cycles×hybridization temperature] to test in order to determine the optimum amplification conditions. For example, in the context of example 1 below the optimum temperature of hybridization of the primers is, in the presence of 10% DMSO, between 50 and 52° C. (limits inclusive), which leads to those skilled in the art having to test only the nine combinations [22-24 cycles×50-52° C.] to determine the optimum operating conditions. The composite primers according to the invention thus have optimum amplification condition ranges which are narrow, and can be predicted in advance.

It should be noted that the presence of primer tags according to the invention makes it possible to use hybridization conditions which, in the course of the 1^{st} and of the 2^{nd} amplification cycle, are at the limit of stability of the pairing of the composite primer on the target. There results therefrom a considerable gain in terms of specificity.

In addition, the composite primers according to the invention preserve the quantitative precision of the amplification results, even when they are used on a nucleic acid or a mixture of nucleic acids which corresponds to a chromosomal region, to a complete chromosome, or even to total genomic DNA.

The tags and composite primers according to the invention, due to their qualities for implementing a quantitative amplification in general, and a multiplex quantitative amplification in particular, find many applications of interest in the biotechnological, medical and veterinary fields.

The method of amplification according to the invention, due to its quantitative nature, can be used to assay certain nucleotide targets in a sample. It can, for example, be applied to assaying the number of copies of transgenes in transgenic animals (rodents, bovines, ovines, for example) which are used to set up the production of substances of biomedical or biotechnological interest, or intended for this production.

It can, for example, be applied, in the field of animal or plant biology, to determining the number of copies of genes, or the presence/absence of certain genes, or the fine characteristics of certain chromosomal regions, which can confer agronomic advantages and which may be involved in the selection or maintenance of certain crosses.

The method of amplification according to the invention can in particular be used to detect genomic rearrangements. The present application is thus directed towards any method for determining the presence or absence of at least one genomic rearrangement within a genetic material B relative to a reference genetic material A, characterized in that:
at least one nucleotide target which constitutes a marker for the rearrangement(s) sought is selected, and in that
a method of amplification according to the invention is applied to said genetic material B, using, for each target selected, a pair of composite primers which is chosen from a plurality of pairs of composite primers according to the invention, and which is suitable for the amplification of this target from the genetic material B,
   said material B being considered as exhibiting said genetic rearrangement when the result of amplification of said at least one marker target, obtained from the material B, is significantly different from that which is obtained from the reference material A under the same conditions, and
   said material B being considered as not exhibiting said genetic rearrangement when the result of amplification of said at least one marker target, obtained from the material B, is not significantly different from that which is obtained from the reference material A under the same conditions.

This method can be applied to cases of genomic rearrangements which are in fact gene rearrangements, but also, and particularly advantageously, to cases of chromosomal rearrangements. Notably, such a method makes it possible to detect chromosomal rearrangements which, until now, were considered to be cryptic (not detectable by standard karyotype techniques), such as the subtelomeric rearrangements involved in many unexplained cases of mental retardation. For applications in the medical field, said genetic material B comprises at least one human gene: it may, for example, be a sample which contains a human chromosomal region, a complete human chromosome or total human genomic DNA. Such a method can, for example, be applied to the detection of gene rearrangements involved in diseases with Mendelian determinism, such as familial forms of breast cancer, hereditary non-polyposis colorectal cancer (HNPCC) or infantile spinal muscular atrophy, or of chromosomal rearrangements possibly involved in diseases with non-Mendelian determinism, such as schizophrenia or unexplained mental retardation.

The method of the invention has also been successfully applied to the detection of gene rearrangements involved the Von Hippel Lindau cancer syndrome (VHL), in multiple endocrine neoplasia (MEN1), in neurofibromatosis (NF2), in retinoblastoma (RB1), in the Peutz-Jeghers syndrome (STK11) and in the Sotos syndrome (NSD1).

It has further been successfully applied to the detection of chromosomal rearrangements involved in the Smith Magenis syndrome (region 17p11) and of those involved in beta thalassemias (region 11p15.5)

The method according to the invention which is capable of determining the presence or absence of at least one genomic rearrangement within a genetic material B relative to a reference genetic material A therefore constitutes a novel method for diagnosing such diseases.

Preferentially, in accordance with the present invention, use is made, as amplification primers to be combined with the tags according to the invention, of the amplification primers which target short nucleotide fragments (from 90 to 500 bp, preferentially from 90 to 350 bp, very preferentially from 90 bp to 300 bp) chosen on various exons representative of the supposed rearrangement.

Compared to the techniques of the prior art which are suitable for detecting chromosomal rearrangements, the method according to the invention has many advantages. For example, with the method according to the invention, it is not necessary to carry out a pre-culture before analysis (unlike the FISH method), nor is it necessary to use specific revealing equipment (unlike CGH); it can be used directly on a sample taken, and requires only conventional means commonly used for carrying out fluorescent PCR. The method according to the invention is also very powerful in terms of sensitivity of detection, while at the same time being technically very simple and very inexpensive compared to the techniques of the prior art. Another major asset lies in its evolutive nature: the tags of amplification primers according to the invention can be coupled to any desired amplification primer, thus conferring on them the desirable kinetic homogeneity.

The method according to the invention for determining the presence or absence of at least one genomic rearrangement is more particularly suitable for determining the presence or absence of genomic rearrangements which involve a loss of genetic material in the heterozygous state.

The tags and composite primers according to the invention also make it possible to determine the limits of any detected genomic rearrangement. For a given patient, it is therefore possible, by virtue of the present invention, to determine the exact extent of the rearrangements exhibited by his or her genetic material, and thus to give a much more precise diagnosis, with possible consequences for the prognosis and treatment. The present application is thus directed to any method for determining at least one of the limits of one or more genomic rearrangement(s) which has (have) been detected within a genetic material B by comparison with a reference genetic material A, characterized in that:
a) a candidate region within which said at least one limit is potentially located is chosen,
b) for each rearrangement, a set of nucleotide targets is chosen, among which at least one is chosen to constitute a marker for this rearrangement, the other target(s) being chosen on both sides or on one or other sides of this marker target inside the candidate region chosen in step a) so as to cover the extent of this candidate region,
c) a method of amplification according to the invention is applied to said genetic material B, using, for each target of said chosen set, at least one pair of composite primers which is chosen from a plurality of pairs of composite primers according to the invention, and which is suitable for amplifying this target from said genetic material A,
d) for each target, the intensity of amplification thus obtained from said genetic material B is measured, and it is compared to the control intensity which is obtained for this same target under the same conditions but by applying said method of amplification to said reference genetic material A,
e) it is determined whether, within the chosen set of targets, at least one target is amplified with an intensity not significantly different from the control intensity, and, if this is not the case, steps a) to e) are repeated while broadening the candidate region chosen in step a),
   said at least one limit of the or of each one of the rearrangements within said genetic material B being considered to be within a zone between:
the position of the marker target for said rearrangement, and
the position of the target which has been amplified with an intensity not significantly different from the control intensity or, if there are several of them, with that which is closest to said marker target,
f) if desired, the precision of determination of said limit is refined, by gradually walking into the zone determined in step e) above, by repeating steps a) to e), choosing as candidate region in step a) the zone identified in the immediately preceding step e), and choosing in step b) a set of nucleotide targets which covers this zone identified in step e).

The present application is also directed towards any genomic rearrangement map which can be obtained using this method, by determining the limits of at least one genomic rearrangement and recording this (these) limit(s) on a gene or chromosomal map. For medical applications, said genetic material will be of human origin, which makes it possible to draw up maps of human genomic rearrangements. Such maps fall within the field of the present application. Just as for the method of amplification itself, the genomic rearrangements in question can be gene rearrangements, just as they may be chromosomal, including cryptic, rearrangements.

The method for determining the limits of one or more genomic rearrangement(s) according to the invention also makes it possible to detect, and optionally to isolate, the gene(s) involved in a genetic disease. The present application is therefore directed towards any method for identifying, and optionally isolating, at least one gene involved in a genetic disease, characterized in that:
the method for determining the presence or absence of at least one genomic rearrangement according to the invention is carried out on a genetic material B derived from organisms exhibiting said genetic disease, a genomic material which is comparable but derived from control organisms serving as reference genomic material A, so as to detect the rearrangement(s) present in the material B relative to the material A, and in that
the gene(s) affected by the detected rearrangement(s) is (are) identified, and optionally isolated,
   this (these) identified and optionally isolated gene(s) corresponding to the gene (s) liable to be involved in said genetic disease.

This method for identifying, and optionally isolating, at least one gene liable to be involved in a genetic disease is of particular value for assaying and detecting (presence/absence) genes involved in the genetic susceptibility to developing diseases, for example infectious diseases, both in non-human animals and in humans.

The method for determining the presence or absence of one or more genomic rearrangement(s) in accordance with the invention, and also the method according to the invention for determining the limits of one or more given genomic rearrangement(s), therefore constitute tools of choice for diagnosing a disease associated with a gene or chromosomal rearrangement, for genetic counselling (prenatal genetic counselling, estimation of viability of an existing rearrangement, estimation of the risks of transmission of this arrangement, determination of the causes and consequences of an observed or predicted Mendelian disease, estimation of the risks of infertility or of spontaneous abortion, determination of the means of preventing, compensating, improving the clinical condition).

The present application is therefore directed towards any method for diagnosing a genetic disease from which an individual might suffer, or for estimating a propensity for this individual to develop such a disease, characterized in that the method for determining the presence or absence of at least one genomic rearrangement according to the invention is used and applied to a representative sample of genetic material from said individual, and in that

said diagnosis is considered to be positive, or, where appropriate, said propensity is considered to be high, when said at least one genetic rearrangement is determined as being present in said sample and, conversely,

said diagnosis is considered to be negative, or, where appropriate, said propensity is considered to be low, when said at least one genomic rearrangement is determined as being absent from said sample. Said genomic rearrangement preferably is a rearrangement which has been determined as associated with the pathogenicity of the disease within other members of the individuals family.

Such a method can be used in vitro, on a sample of genetic material representative of said genetic disease, for example on a biological sample taken from said individual, and in particular from a human, for whom a disease with a genetic component is suspected (for example on the basis of family history). Determination of the limits of the possible genomic rearrangements detected should make it possible to refine the diagnosis and, optionally, the vital or pathological prognosis, or even to adjust the therapy as a consequence.

The individual which is the subject of said diagnosis or prognosis may, for example, be an animal, such as a mammal, an ovine, a bovine or a rodent. Advantageously, this animal may be a human.

The prognostic method is particularly suitable when a genomic rearrangement involved in said genetic disease has already been detected in the individual's family. In this case, said propensity is considered to be low when said at least one genomic rearrangement is determined as being absent from an individual belonging to a family which carries the previously detected genomic rearrangement.

The present application is also directed towards any kit for carrying out a method of amplification according to the invention, and/or a method for determining the presence or absence of gene or chromosomal rearrangements according to the invention, and/or a method for determining the limits of one or more gene or chromosomal rearrangement(s) according to the invention, and/or a method for identifying at least one gene involved in a genetic disease according to the invention, and/or a diagnostic or prognostic method according to the invention. A kit according to the invention comprises at least one pair of composite primers according to the invention, and/or at least one composite primer according to the invention, and/or at least one tag according to the invention, optionally combined with an amplification primer and/or with a label for detecting nucleotide products. It may advantageously comprise a plurality of pairs of composite primers according to the invention.

The present invention is illustrated by the following examples, given purely by way of illustration: they in no way limit it.

In these examples, reference is made to the following figures:

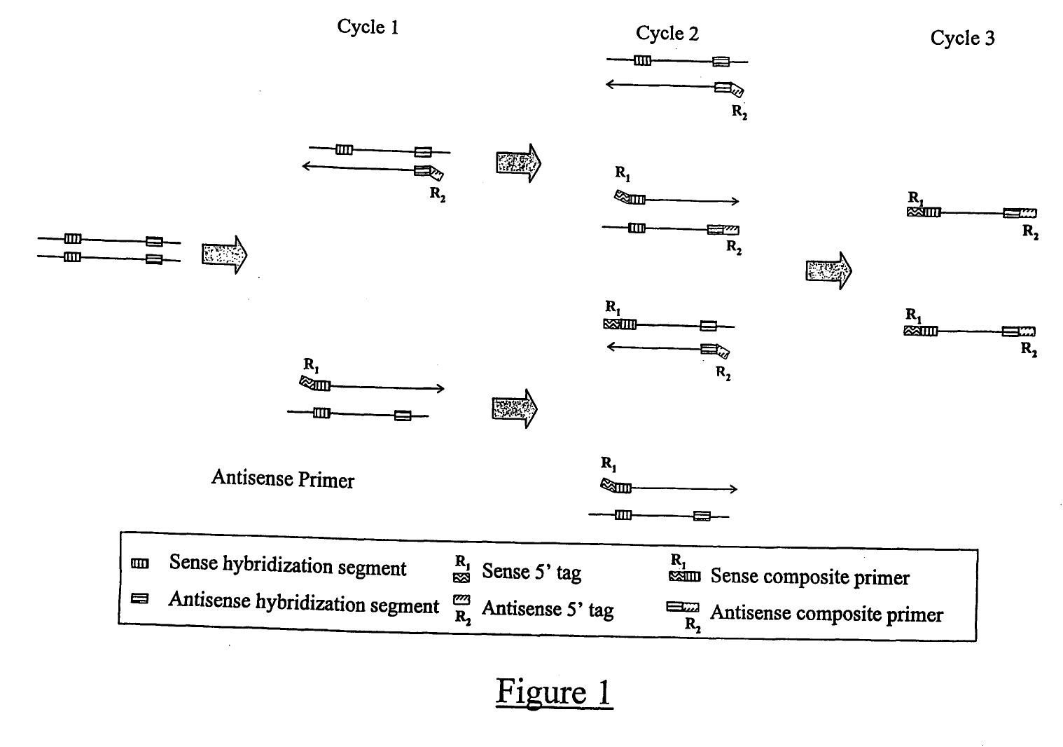

FIG. 1 gives a diagrammatic representation of a PCR carried out using composite primers according to the invention: after two amplification cycles, a neo-matrix is formed by incorporation of the 5′ tags (priming phase),

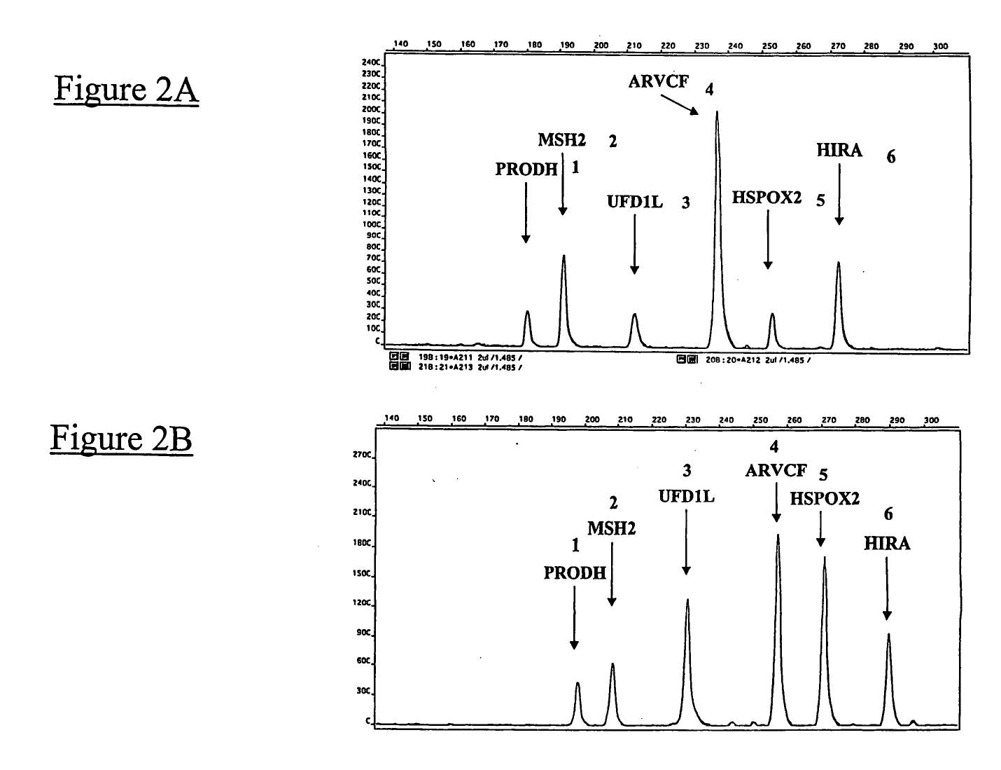

FIGS. 2A and 2B provide, for six fragments corresponding to the PRODH, UFD1L, ARVCF, HSPOX2, HIRA and MSH2 genes, a comparison of the fluorescence profile obtained at a given number of PCR cycles (here 22) by following the method according to the invention (FIG. 2B: fluorescent multiplex PCR carried out using composite primers according to the invention, and in the presence of DMSO), relative to the fluorescence profile obtained by following a method of the prior art (FIG. 2A: comparable fluorescent multiplex PCR, but carried out using conventional primers without tag, and in the absence of DMSO),

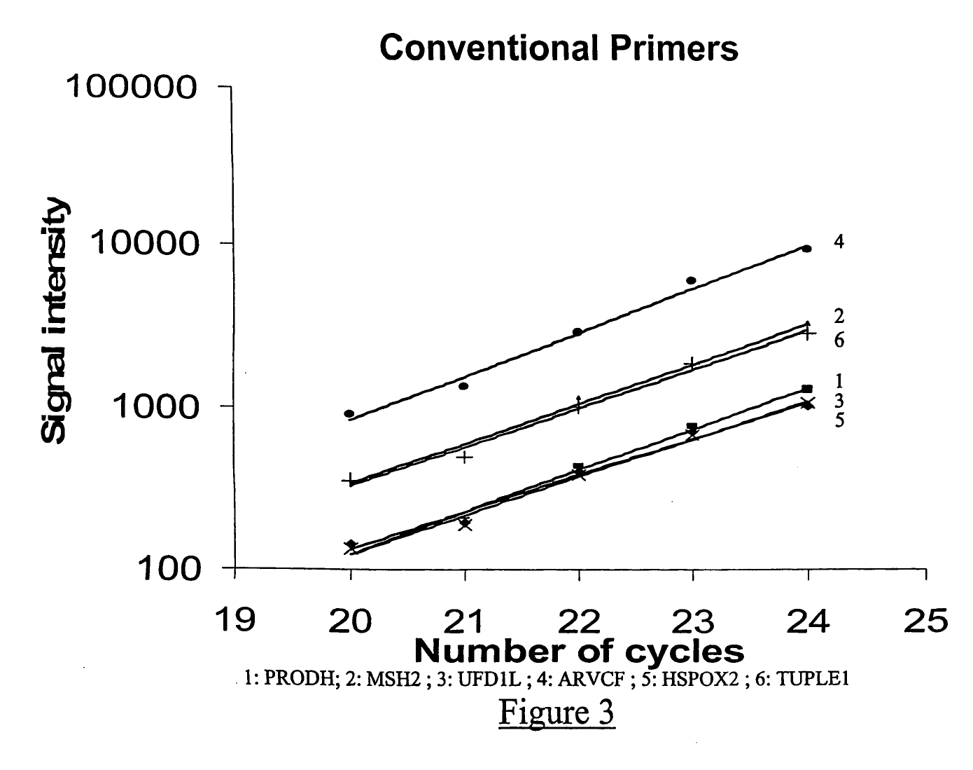

FIGS. 3, 4, 5 and 6 show, by way of comparison, semi-logarithmic plots representative of the signal intensities measured during the amplification of six short fluorescent fragments (corresponding to the PRODH, UFD1L, ARVCF, HSPOX2, HIRA and MSH2 genes) as a function of the number of PCR cycles (plot 1=PRODH; plot 2=MSH2; plot 3=UFD1L; plot 4=ARVCF; plot 5=HSPOX2; plot 6=TUPLE1):

FIG. 3 (prior art): multiplex PCR of short fluorescent fragments carried out using conventional primers (=primers without tag), and in the absence of DMSO,

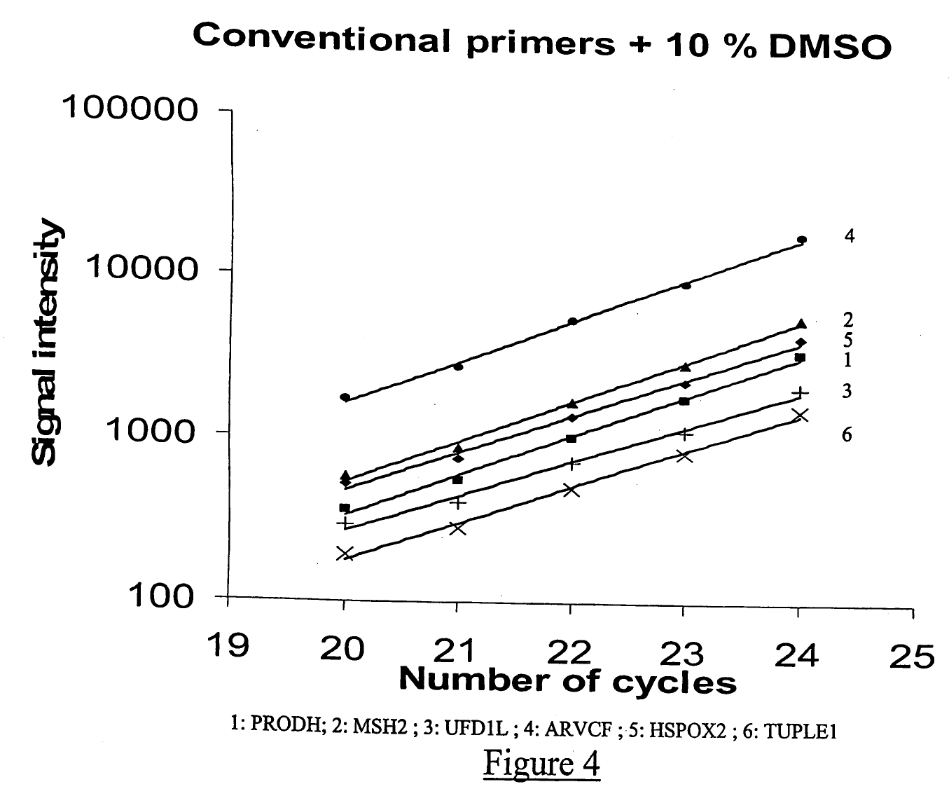

FIG. 4 (prior art): multiplex PCR of short fluorescent fragments carried out using conventional primers (=primers without tag), and in the presence of 10% DMSO,

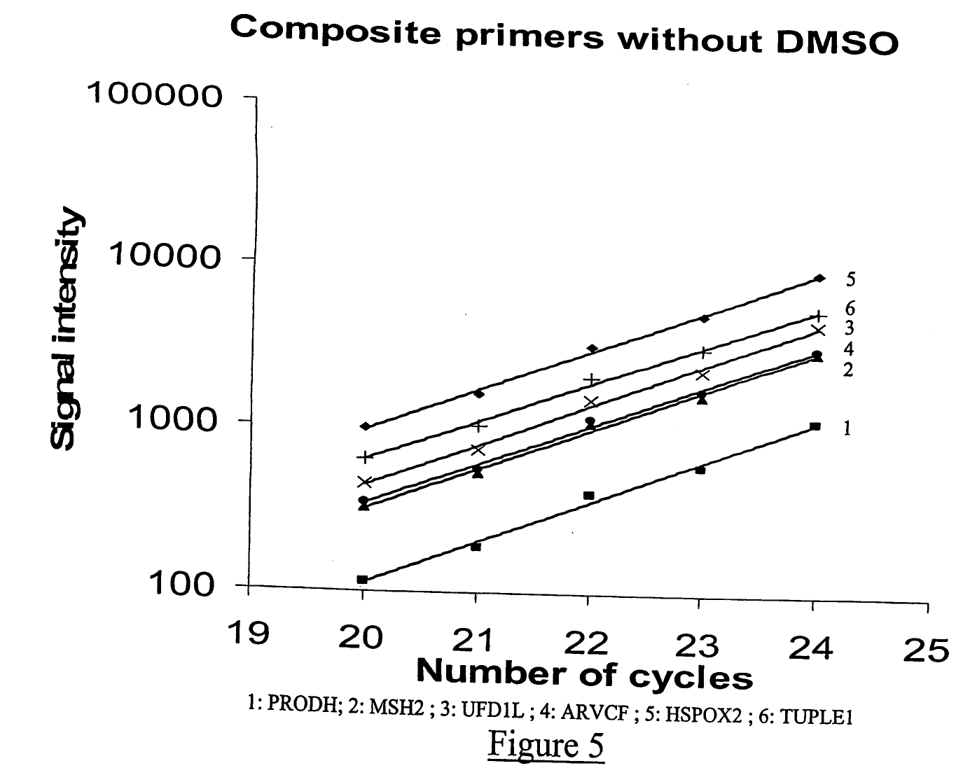

FIG. 5 (present invention): multiplex PCR of short fluorescent fragments carried out using composite primers according to the invention (=with tags according to the invention), and in the absence of DMSO,

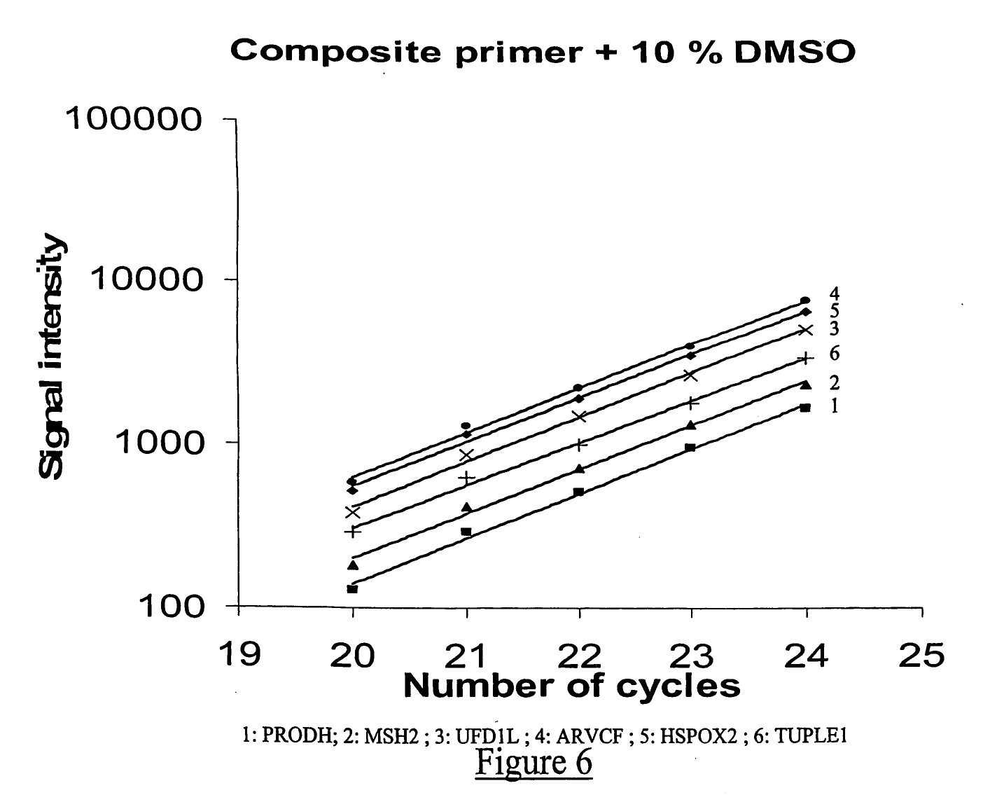

FIG. 6 (preferred embodiment of the present invention): multiplex PCR of short fluorescent fragments carried out using composite primers according to the invention (=with tags according to the invention), and in the presence of 10% DMSO,

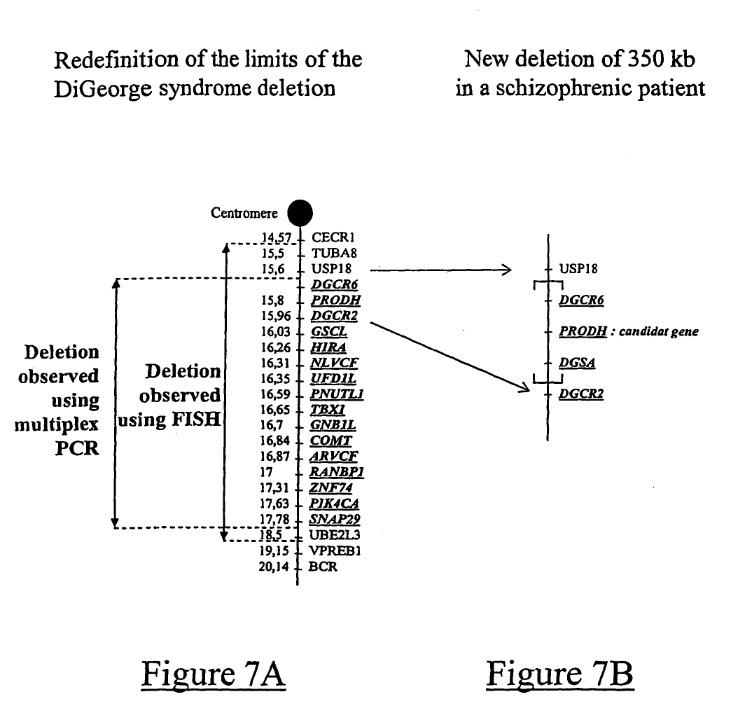

FIG. 7A illustrates the search for a gene involved in schizophrenia and the determination of the limits of a chromosomal rearrangement: this figure gives chromosomal region 22q11 (which, in DiGeorge syndrome, undergoes a deletion), and shows the 22 genes of this region on which short exon fragments were chosen and then amplified in multiplex in accordance with the invention, which made it possible to redefine the limits of the DiGeorge syndrome deletion (the 16 genes affected by the deletion are marked in bold underlined),

FIG. 7B illustrates the determination of the limits of a 350 kb deletion in a schizophrenic patient, and the identification of a candidate gene for involvement in schizophrenia (PRODH),

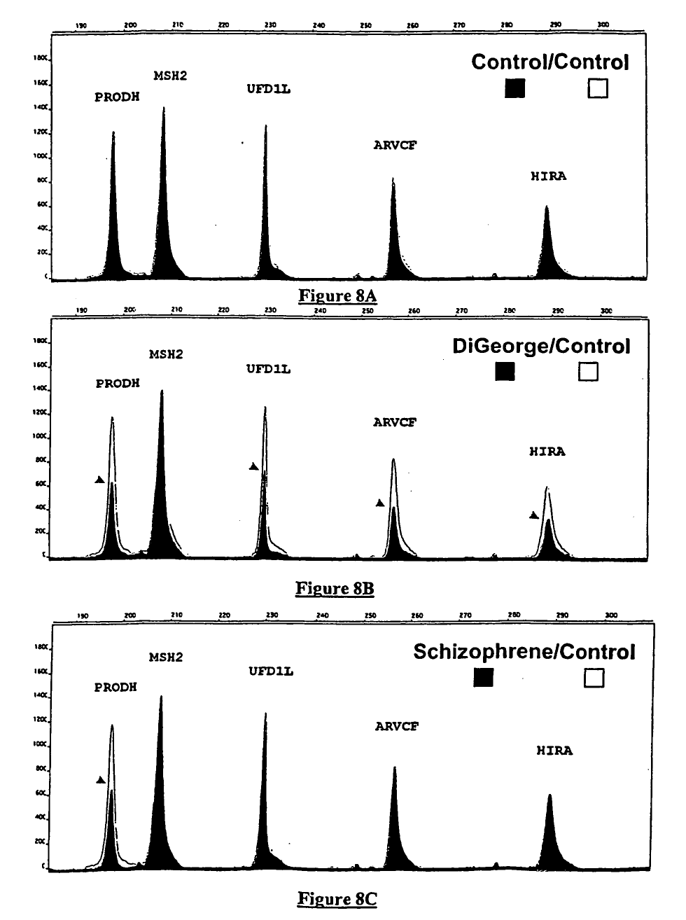

FIGS. 8A, 8B and 8C illustrate results of determination of 22q11 chromosomal rearrangements which were obtained using a multiplex PCR in accordance with the invention:

FIG. 8A: profile of amplification of short fragments chosen within genes of a patient free of 22q11 rearrangement;

FIG. 8B: profile of amplification of short fragments chosen within genes of a patient suffering from DiGeorge syndrome, compared to that of an unaffected patient;

FIG. 8C: profile of amplification of short fragments chosen within genes of a patient suffering from schizophrenia, compared to that of an unaffected patient,

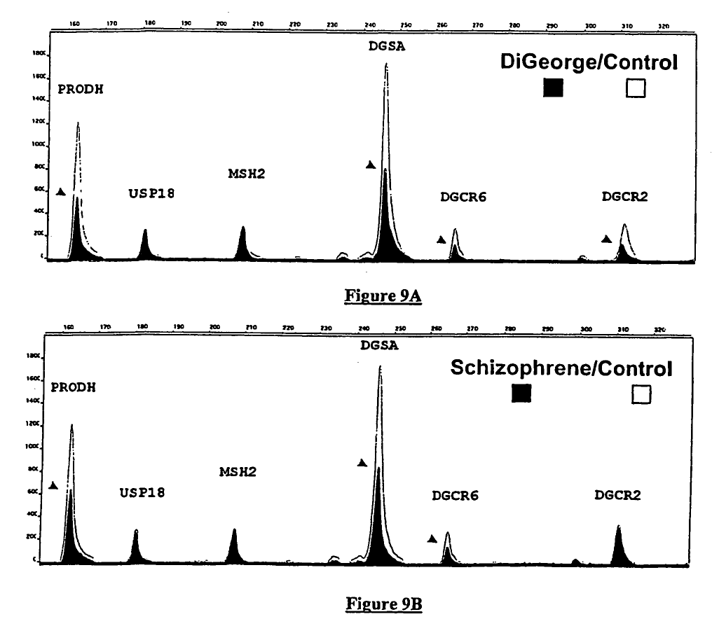

FIGS. 9A and 9B illustrate results of determination of chromosomal rearrangements which were obtained using a multiplex PCR in accordance with the invention, using the targets indicated in FIG. 7B:

FIG. 9A: profile of amplification of short fragments chosen within genes of a patient suffering from DiGeorge syndrome, compared to that of an unaffected patient;

FIG. 9B: profile of amplification of short fragments chosen within genes of a patient suffering from schizophrenia, compared to that of an unaffected patient.

### EXAMPLE 1

### Demonstration of the Quantitative Level of Precision Attained for the Detection of Chromosomal Rearrangements (Chromosome 22)

In order to illustrate some of the advantages of the method according to the invention compared to the methods which could previously have been carried out, fluorescent multiplex PCR experiments were carried out under comparable conditions, but varying two factors: the type of primers used (primers in accordance with the present invention, or primers corresponding to the practice of the prior art), and the presence or absence of DMSO (dimethyl sulphoxide).

By way of illustration, it was chosen, in these experiments, to target the chromosomal rearrangements of a part of the long arms of chromosome 22, which are involved in DiGeorge polymalformation syndrome.

In order to detect these chromosomal rearrangements, it was chosen to target five genes located on human chromosome 22 (PRODH, UFD1L, ARVCF, HSPOX2 and HIRA genes), and also a fragment used as an internal non-rearranged control for the multiplex PCR and derived from the MSH2 gene which is located on human chromosome 2. Short polynucleotide fragments constituting molecular indicators of the possible chromosomal rearrangements were selected within the sequence of each one of these genes. Based on the sequence of these short fragments, primers were constructed which make it possible to amplify them specifically (=primers constructed in accordance with the prior art). The tags according to the invention were added to these primers, in the 5′ position, thus forming composite primers according to the invention. The two types of set of primers (conventional set without tag, and set of primers according to the invention with specific tags in the 5′ position) are tested under comparable operating conditions.

The multiplex PCRs are carried out using normal genomic DNA extracted from total blood taken from unaffected individuals, and simultaneously amplifying the various short fragments specific for these six genes (multiplex PCR). Two operating conditions are tested: presence of DMSO (10%) or absence of DMSO.

A/ PCR Operating Conditions

The 25 μL reaction volume of the PCR used in this example is made up in the following way:
75 mM Tris HCl, pH 8.8
20 mM (NH₄)₂SO₄
0.01% Tween® 20
1.5 mM MgCl₂
200 μM dNTPs (deoxyribonucleoside triphosphates) for the PCRs carried out in the presence of DMSO, DMSO is added at a final concentration of 10% all the primers are present at the same concentration of 0.3 μM.

Taq polymerase (marketed under the name “Thermoprime Plus DNA Polymerase”, ABgene®) is used in this example at a dose of 1.2 units per tube. A unit of this enzyme is defined as the amount which incorporates into a PCR product 10 nanomoles of dNTP in 30 min at 74° C.

The initial amount of DNA is fixed at 100 ng of genomic DNA extracted from whole blood using the “QIAmp® DNA Blood Mini Kit” from Qiagen, and then accurately assayed using the picogreen® dsDNA quantitation reagent system (Molecular Probes).

The PCR reaction is carried out in an MJResearch PTC100 96 A/V thermocycler.

The composite primers according to the invention which were used to amplify each one of the six short gene fragments (peak 1 to peak 6) are given in Table 1 below. Each one of these composite primers consists (from 5′ to 3′) of a tag of 10 nucleotides according to the invention and of a hybridization segment. The sense composite primers also carry labelling in the 5′ position (here, 6-FAM fluorescent labelling).

The sequence of the sense tag according to the invention is:
CGT TAG ATA G (SEQ ID NO:1 according to the invention).

The sequence of the antisense tag according to the invention is:
GAT AGG GTT A (SEQ ID NO:2 according to the invention).

The hybridization segments used are:
for the short fragment of the PRODH gene (peak 1), ACTCCATCTCCTTGTGCTCT (SEQ ID NO:3) for the sense primer, and CGCTATTCAACAAGCTCATG for the antisense primer (SEQ ID NO:4),
for the short fragment of the MSH2 gene (peak 2), GGTAAAACACATTCCTTTGG (SEQ ID NO:5) for the sense primer, and ATATGTGAGCTTCCATTGGT for the antisense primer (SEQ ID NO:6),
for the short fragment of the UFD1L gene (peak 3), ATGTTTAACAACCGCCAGCA (SEQ ID NO:7) for the sense primer, and TCTTCCTTTCAGATGATGCAGA for the antisense primer (SEQ ID NO:8),
for the short fragment of the ARVCF gene (peak 4), GACATGGTGCTGTGTGTGAGC (SEQ ID NO:9) for the sense primer, and TCCGCCTTTAGAAGTCCAAGT for the antisense primer (SEQ ID NO:10),
for the short fragment of the HSPOX2 gene (peak 5), TGAAGCTGTGTGGCTGAAAC (SEQ ID NO:11) for the sense primer, and TAGCCAGGGTGTCTCAAAGA for the antisense primer (SEQ ID NO:12),
for the short fragment of the HIRA gene (peak 6), TACCAGTCATCGGGCAGAAC (SEQ ID NO:13) for the sense primer, and AATGTCAGAGGCAGGACACAG for the antisense primer (SEQ ID NO:14).

The composite primers according to the invention used here and producing peaks 1-6 shown in FIG. 2B therefore have the sequences given in Table 1 below:
peak 1 (PRODH): SEQ ID NO:15 and 16 for the sense and antisense composite primers respectively,
peak 2 (MSH2): SEQ ID NO:17 and 18 for the sense and antisense composite primers respectively,
peak 3 (UFD1L): SEQ ID NO:19 and 20 for the sense and antisense composite primers respectively,
peak 4 (ARVCF): SEQ ID NO:21 and 22 for the sense and antisense composite primers respectively,
peak 5 (HSPOX2): SEQ ID NO:23 and 24 for the sense and antisense composite primers respectively,
peak 6 (HIRA): SEQ ID NO:25 and 26 for the sense and antisense composite primers respectively.

The 5′ end of each sense primer (SEQ ID NOS: 15, 17, 19, 21, 23 and 25) carries a fluorescent label (in the examples given here, 6-FAM labelling available for example from the company Applied Biosystems).

The conventional primers used by way of comparison comprise no tag. They therefore consist (from 5′ to 3′) of a fluorescent label (6-FAM) and of the hybridization segment: SEQ ID NO: 3, 5, 7, 9, 11, 13 for the sense primers, and SEQ ID NO: 4, 6, 8, 10, 12, 14 for the antisense primers which correspond to them respectively.

TABLE 1

| | | Size | | |
|---|---|---|---|---|
| | Gene | amplified | | |
| | tar- | (composite | | |
| Peak | geted | primer) | Labelled sense primer | Antisense primers |
| Peak 1 | PRODH | 190 bp | 5′ 6-FAM-CGTTAGATAGACTCCATCTCCTTGTGCTCT 3′ | 5′ GATAGGGTTACGCTATTCAACAAGCTCATG 3′ |
| Peak 2 | MSH2 | 207 bp | 5′ 6-FAM-CGTTAGATAGGGTAAAACACATTCCTTTGG 3′ | 5′ GATAGGGTTAATATGTGAGCTTCCATTGGT 3′ |
| Peak 3 | UFD1L | 228 bp | 5′ 6-FAM-CGTTAGATAGATGTTTAACAACCGCCAGCA 3′ | 5′ |
| | | | | GATAGGGTTATCTTCCTTTCAGATGATGCAGA 3′ |
| Peak 4 | ARVCF | 254 bp | 5′ | 5′ |
| | | | 6-FAM-CGTTAGATAGGACATGGTGCTGTGTGTGAGC 3′ | GATAGGGTTATCCGCCTTTAGAAGTCCAAGT 3′ |
| Peak 5 | HSPOX2 | 270 bp | 5′ 6-FAM-CGTTAGATAGTGAAGCTGTGTGGCTGAAAC 3′ | 5′ GATAGGGTTATAGCCAGGGTGTCTCAAAGA 3′ |
| Peak 6 | HIRA | 290 bp | 5′ 6-FAM-CGTTAGATAGTACCAGTCATCGGGCAGAAC 3′ | 5′ |
| | | | | GATAGGGTTAAATGTCAGAGGCAGGACACAG 3′ |

The following protocols were compared.

Multiplex PCR of Short Fluorescent Fragments with Conventional Primers Without DMSO (FIG. 3):

After denaturation for 5 minutes at 94° C., 18 to 27 PCR cycles are carried out according to the characteristics below, followed by an elongation step of 5 minutes at 72° C.;

10 seconds at 94° C.
15 seconds at 54° C.
20 seconds at 72° C.

Multiplex PCR of Short Fluorescent Fragments with Conventional Primers+10% DMSO (FIG. 4):

After denaturation for 5 minutes at 94° C., 18 to 27 PCR cycles are carried out according to the characteristics below, then followed by an elongation step of 5 minutes at 72° C.;
10 seconds at 94° C.
15 seconds at 50° C.
20 seconds at 72° C.

Multiplex PCR of Short Fluorescent Fragments with Composite Primers (5′ tags) Without DMSO (FIG. 5):

After denaturation for 5 minutes at 94° C., 18 to 27 PCR cycles are carried out according to the characteristics below, then followed by an elongation step of 5 minutes at 72° C.;
10 seconds at 94° C.
15 seconds at 54° C.
20 seconds at 72° C.

Multiplex PCR of Short Fluorescent Fragments with Composite Primers (5′ tags) and 10% DMSO (FIG. 6):

After denaturation for 5 minutes at 94° C., 18 to 27 PCR cycles are carried out as described below and are followed by an elongation step of 5 minutes at 72° C.;
10 seconds at 94° C.
15 seconds at 50° C.
20 seconds at 72° C.

The fluorescence intensities obtained using each one of the protocols described above, for a number of cycles of between 20 and 24, are measured after separation and quantitative analysis in the Applied Biosystems 377 DNA sequencer, used in fragment analysis mode (Genescan™ program). The results, expressed as signal intensities measured as a function of the number of cycles, are converted to a semi-logarithmic scale, in order to compare the amplification kinetics for the six various fragments. FIGS. 3 to 6 illustrate these results. Each DNA segment amplified in the multiplex PCR is represented by different lines: plot 1=PRODH; plot 2=MSH2; plot 3=UFD1L; plot 4=ARVCF; plot 5=HSPOX2; plot 6=TUPLE1.

FIG. 3 (prior art): conventional primers (=primers without tag), and in the absence of DMSO,

FIG. 4 (prior art): conventional primers (=primers without tag), and in the presence of 10% DMSO,

FIG. 5 (present invention): composite primers according to the invention (=with tags according to the invention) and in the absence of DMSO,

FIG. 6 (preferred embodiment of the present invention): composite primers according to the invention (=with tags according to the invention), and in the presence of 10% DMSO.

It may be noted that the amplification kinetics for the six various fragments (expressed in FIGS. 3 to 6 according to a semi-logarithmic scale) are clearly more homogeneous by following the method according to the invention than by following a method of the prior art: the kinetics obtained with the composite primers according to the invention (cf. FIG. 5—without DMSO—, and FIG. 6—with DMSO—) are more homogeneous than those obtained with the conventional primers without tag (cf. FIG. 3—without DMSO—, and FIG. 4—with DMSO—). It is also possible to assess the particularly notable level of homogeneity attained when the use of composite primers according to the invention is combined with that of DMSO.

The fluorescence profiles obtained at a given number of cycles themselves also show that the intensity of the peaks corresponding to the six amplified fragments (peak 1 to peak 6) is clearly more homogeneous in the profile which is obtained using the tags according to the invention and 10% DMSO (cf. FIG. 2B, fluorescence profile at 22 cycles), than when neither a tag nor DMSO was used (cf. FIG. 2A, fluorescence profile at 22 cycles).

These results demonstrate that, by virtue of the 5′ tags according to the invention, the composite primers can all be introduced at the same concentration, while at the same time producing homogeneous amplification kinetics, whatever the fragments amplified: a quantitative level of precision is therefore attained by virtue of these 5′ tags without it being necessary to search for the appropriate concentrations for each one of the primers used. The quantitative fidelity is even better when the use of composite primers is combined with that of DMSO.

The tags according to the invention can be used with any hybridization segment, and the inventors have been able to note that the performances remain stable even when about twelve different segments are amplified in multiplex.

As illustrated below, the combined use of composite primers according to the invention and DMSO also has the advantage of limiting, for any new combination of segments to be amplified in the same multiplex PCR, the tests for optimizing the number of cycles and temperature (with the tags according to the invention, and using DMSO, the optimum number of cycles is between 22 and 24, independently of the hybridization segments used, and by choosing hybridization segments with close Tm values, the temperature range to be tested remains limited).

B/ Optimization for Each New PCR Multiplex:

Choice of the Number of Cycles and of the Hybridization Temperature

While keeping constant the conditions established above for the PCR which uses the composite primers and DMSO (preferred embodiment of the invention), the setting up for each new multiplex PCR requires only 9 tests to be carried out, which tests are aimed at choosing the best combination of temperature and number of cycles (cf. Table 2 below).

For the amplifications shown in this example, the optimum temperature, in the presence of 10% DMSO, is in between 50 and 52° C., and the optimum number of cycles is between 22 and 24.

TABLE 2

| Number | Temperature | | |
|---|---|---|---|
| of cycles | 50° C. | 51° C. | 52° C. |
| 22 | Test 1 | Test 2 | Test 3 |
| 23 | Test 4 | Test 5 | Test 6 |
| 24 | Test 7 | Test 8 | Test 9 |

### EXAMPLE 2

### Determination of the Limits of a Genomic Rearrangement, and Identification, Using the Quantitative Multiplex PCR Method According to the Invention, of a Gene Involved in a Genetic Disease

A. Materials and Methods:

1. PCR Operating Conditions:

The PCR operating conditions used in this example correspond to those described in Example 1, namely:

The 25 μL reaction volume of the PCR used in this example is made up in the following way:
75 mM Tris HCl, pH 8.8
20 mM (NH₄)₂SO₄
0.01% Tween® 20
1.5 mM MgCl₂
200 μM dNTPs (deoxyribonucleoside triphosphates) for the PCRs carried out in the presence of DMSO, DMSO is added at a final concentration of 10% all the primers are present at the same concentration of 0.3 μM.

Taq polymerase (marketed under the name “Thermoprime Plus DNA Polymerase”, ABgene®) is used in this example at a dose of 1.2 units per tube. A unit of this enzyme is defined as the amount which incorporates into a PCR product 10 nanomoles of dNTP in 30 min at 74° C. The initial amount of DNA is fixed at 100 ng of genomic DNA extracted from whole blood using the “QIAmp® DNA Blood Mini Kit” from Qiagen, and then accurately assayed using the “picogreen® dsDNA quantitation reagent system” (Molecular Probes).

The PCR reaction is carried out in an MJResearch PTC100 96 A/V thermocycler.

2. Composite Primers for Determining the Limits of the Deletion of Region 22q11 (DiGeorge Syndrome):

The tags SEQ ID NO:1 and SEQ ID NO:2 according to the invention were used to form the sense and antisense composite primers, respectively, having the following hybridization segments: for the short fragment of the PRODH gene, ACTCCATCTCCTTGTGCTCT (SEQ ID NO:3) for the sense primer, and CGCTATTCAACAAGCTCATG for the antisense primer (SEQ ID NO:4), for the short fragment of the MSH2 gene, GGTAAAACACATTCCTTTGG (SEQ ID NO:5) for the sense primer, and ATATGTGAGCTTCCATTGGT for the antisense primer (SEQ ID NO:6), for the short fragment of the UFD1L gene, ATGTTTAACAACCGCCAGCA (SEQ ID NO:7) for the sense primer, and TCTTCCTTTCAGATGATGCAGA for the antisense primer (SEQ ID NO:8), for the short fragment of the ARVCF gene, GACATGGTGCTGTGTGTGAGC (SEQ ID NO:9) for the sense primer, and TCCGCCTTTAGAAGTCCAAGT for the antisense primer (SEQ ID NO:10), for the short fragment of the HIRA gene, TACCAGTCATCGGGCAGAAC (SEQ ID NO:13) for the sense primer, and AATGTCAGAGGCAGGACACAG for the antisense primer (SEQ ID NO:14).

The composite primers according to the invention used here therefore have the sequences which were described in Table 1 above.
PRODH: SEQ ID NO:15 and 16 for the sense and antisense composite primers respectively,
MSH2: SEQ ID NO:17 and 18 for the sense and antisense composite primers respectively,
UFD1L: SEQ ID NO:19 and 20 for the sense and antisense composite primers respectively,
ARVCF: SEQ ID NO:21 and 22 for the sense and antisense composite primers respectively,
HIRA: SEQ ID NO:25 and 26 for the sense and antisense composite primers respectively.

The 5′ end of each sense composite primer (SEQ ID NOS: 15, 17, 19, 21 and 25) carries fluorescent labelling (6-FAM available for example from the company Applied Biosystems).

3. Composite Primers for Identifying a Gene Liable to be Involved in Schizophrenia (Multiplex PCR of the Genomic Region Surrounding the PRODH Gene, cf. FIG. 7B):

The composite primers according to the invention which were used to amplify each one of the six short gene fragments are given in table 3 below.

Each one of these composite primers consists (from 5′ to 3′) of a tag of 10 nucleotides according to the invention and of a hybridization segment. The sense composite primers also carry labelling (here, 6-FAM fluorescent labelling).

The sequence of the sense tag according to the invention is:
CGT TAG ATA G SEQ ID NO:1 according to the invention.

The sequence of the antisense tag according to the invention is:
GAT AGG GTT A SEQ ID NO:2 according to the invention.

The hybridization segments used are:
for the short fragment of the PRODH gene, CCCTGGTGCGATGGGGT (SEQ ID NO:27) for the sense primer, and GGCACGGCGGGACAAGTAG for the antisense primer (SEQ ID NO:28),
for the short fragment of the USP18 gene, AGTCGTGCTGTCCTGAACG (SEQ ID NO:29) for the sense primer, and TCTTCTTCCTTCTTTTCTTCAA for the antisense primer (SEQ ID NO:30),
for the short fragment of the MSH2 gene, GGTAAAACACATTCCTTTGG (SEQ ID NO:5) for the sense primer, and ATATGTGAGCTTCCATTGGT for the antisense primer (SEQ ID NO:6),
for the short fragment of the DGSA gene, GCATCCTCCTACTCTTCTCCTGG (SEQ ID NO:31) for the sense primer, and AGCCTCCCTCAAATAGGTCT for the antisense primer (SEQ ID NO:32),
for the short fragment of the DGRC6 gene, TGGGGCTAGGAGGTCCCT (SEQ ID NO:33) for the sense primer, and CCTCCCCTTTATGAGACTATCCTA for the antisense primer (SEQ ID NO:34),
for the short fragment of the DGCR2 gene, AGAGGCAGGGAATGAAGAA (SEQ ID NO:35) for the sense primer, and GGGTCACCTTGATATTCACA for the antisense primer (SEQ ID NO:36).

The composite primers according to the invention used here therefore have the sequences given in table 3 below
PRODH: SEQ ID NO:37 and 38 for the sense and antisense composite primers respectively,
USP18: SEQ ID NO:39 and 40 for the sense and antisense composite primers respectively,
MSH2: SEQ ID NO:17 and 18 for the sense and antisense composite primers respectively,
DGSA: SEQ ID NO:41 and 42 for the sense and antisense composite primers respectively,
DGRC6: SEQ ID NO:43 and 44 for the sense and antisense composite primers respectively,
DGCR2: SEQ ID NO:45 and 46 for the sense and antisense composite primers respectively.

The 5′ end of each sense composite primer (SEQ ID NOS: 17, 37, 39, 41, 43 and 45) carries fluorescent labelling (6-FAM available for example from the company Applied Biosystems).

TABLE 3

| | Size | | |
|---|---|---|---|
| Gene | amplified | | |
| tar- | (composite | | |
| geted | primer) | Labelled sense primer | Antisense primers |
| PRODH | 161 bp | 5′ 6-FAM-CGTTAGATAGCCCTGGTGCGATGGGGT 3′ | 5′ GATAGGGTTAGGCACGGCGGGACAAGTAG 3′ |
| USP18 | 180 bp | 5′ 6-FAM-CGTTAGATAGAGTCGTGCTGTCCTGAACG 3′ | 5′ GATAGGGTTATCTTCTTCCTTCTTTTCTTCAA 3′ |
| MSH2 | 207 bp | 5′ 6-FAM-CGTTAGATAGGGTAAAACACATTCCTTTGG 3′ | 5′ GATAGGGTTAATATGTGAGCTTCCATTGGT 3′ |
| DGSA | 243 bp | 5′ 6- | 5′ GATAGGGTTAAGCCTCCCTCAAATAGGTCT 3′ |
| | | FAM-CGTTAGATAGGCATCCTCCTACTCTTCTCCTGG 3′ | |
| DGCR6 | 265 bp | 5′ 6-FAM-CGTTAGATAGTGGGGCTAGGAGGTCCCT 3′ | 5′ GATAGGGTTACCTCCCCTTTATGAGACTATCCTA 3′ |
| DGCR2 | 312 bp | 5′ 6-FAM-CGTTAGATAGAGAGGCAGGGAATGAAGAA 3′ | 5′ GATAGGGTTAGGGTCACCTTGATATTCACA 3′ |

B. Results and Conclusions

To analyse the 22q11 chromosomal region deleted in DiGeorge syndrome, short exon fragments derived from 22 genes located in this region (CECR1, TUBA8, USP18, DGCR6, PRODH, DGCR2, GSCL, HIRA, NLVCF, UFD1L, PNUTL1, TBX1, GNB1L, COMT, ARVCF, RANBP1, ZNF74, PIK4CA, SNAP29, UBE2L3, VPREB1, BCR; cf. FIG. 7A) were amplified simultaneously using four different multiplex PCRs according to the invention. Each one of these multiplex PCRs included 4 to 8 different nucleotide targets of this region and a nucleotide target located outside the region subjected to investigation (in the example given in FIG. 8: an exon of the MSH2 gene, located in chromosome 2).

In order to validate the reproducibility and the specificity of the method, several DNAs from unaffected control individuals (22q11 chromosomal region without rearrangement) were compared over all the 22 nucleotide targets (FIG. 8A shows one of the four multiplex PCRs used).

The DNA of an unaffected control and that of an individual suffering from DiGeorge syndrome and with a deletion observed by the FISH technique were then compared over all the 22 nucleotide targets (FIG. 8B shows the result obtained with one of the four multiplex PCRs).

For each multiplex PCR, the fluorescence profiles obtained from various DNAs were aligned by superposing the fluorescence peaks obtained for the nucleotide target chosen with no rearrangement (in this case: MSH2). FIG. 8B illustrates the heterozygous deletion of the PRODH, UFD1L, ARVCF and HIRA genes represented by the decrease in the peaks for the corresponding nucleotide targets. Among the 18 nucleotide targets included in the other multiplex PCRs, 12 had fluorescence peaks with a height that was also decreased compared to that of the same targets in the non-rearranged controls, while 6 had fluorescence peaks which could be superposed onto those of the non-rearranged control DNAs.

These results therefore showed that the heterozygous deletion of the DiGeorge region comprises the 16 genes represented in italics/bold/underlined in FIG. 7A (DGCR6, PRODH, DGCR2, GSCL, HIRA, NLVCF, UFD1L, PNUTL1, TBX1, GNB1L, COMT, ARVCF, RANBPI, ZNF74, PIK4CA, SNAP29), while the six genes indicated in normal uppercase are not affected by the deletion (CECR1, TUBA8, USP18, UBE2L3, VPREB1, BCR). It should be noted that the limits of the deletion defined by the new method do not coincide with those indicated previously by the FISH method, and that the definition of the limits is more precise with the method described in this invention.

FIG. 7B illustrates the steps which enabled us to focus on a much shorter region for searching for a gene involved in schizophrenia, due to the detection of a 350 kilobase deletion, in a schizophrenic patient. This deletion was discovered, and its limits were located, using the method according to the invention and, specifically, the multiplex PCR shown in FIG. 8C and the two multiplex PCRs shown in FIG. 9. It should be noted that, in FIG. 9A, a non-rearranged DNA is compared with a DNA with DiGeorge deletion (see Scheme 7A), and that, in FIG. 9B, a non-rearranged DNA is compared with the DNA from the schizophrenic patient who exhibited a deletion around the PRODH gene (see FIG. 8C).

In conclusion, in the schizophrenic patient studied, the nucleotide targets corresponding to the USP18 and DGCR2 genes are not affected by the deletion, whereas the targets corresponding to the PRODH, DGSA and DGCR6 genes exhibit a heterozygous deletion revealed by the 50% decrease in the fluorescence intensity of the peaks (FIG. 9B). The functional characteristics of the product of the PRODH gene indicate that it is a very good candidate for involvement in schizophrenia.

The use of the method described in this patent makes it possible to reliably and rapidly carry out many multiplex PCRs which may satisfy the need of those skilled in the art, which consists in reliably and rapidly characterizing the genomic rearrangements of all sizes leading to a loss or a gain of genetic material.

Those skilled in the art may find many technical alternatives to those illustrated and described in the present application. Such alternatives are known to them from the scientific literature in the field of molecular biology in general, of nucleotide sequence amplifications in particular, and of their medical and biotechnological applications. Reference may in particular be made to the basic manuals in the field, such as Maniatis et al. “Molecular Cloning: A Laboratory Manual”, Cold Spring Harbor Laboratory Press, New York; Ausubel P. M. et al. Eds., “Current Protocols in Molecular Biology”; Ehrlich H. A. Ed., “PCR Technology”, Stockton Press, New York (1989). The content of all the documents (scientific publications, patent applications, patents) which are mentioned in the present application is incorporated by way of reference.

1. Method for producing a plurality of pairs of sense and antisense composite primers, said plurality being specially adapted to the quantitative multiplex amplification of a plurality of target nucleotide sequences present in a nucleic acid or a mixture of nucleic acids, wherein each one of said sense or antisense composite primers produced consists:
of a hybridization segment, respectively sense or antisense, which pairs with said nucleic acid or mixture of nucleic acids, so as to constitute a sense or antisense primer for one of the target nucleotide sequences of the plurality targeted, and
of a nucleotide tag which is attached to the 5′ end of said hybridization segment, but which does not pair with said nucleic acid or mixture of nucleic acids,
and, optionally, of a non-nucleotide component,
characterized in that the respective sequences of the sense and antisense composite primers of said plurality of pairs are selected such that:
a) each sense composite primer has, within said plurality, an antisense composite primer with which it forms a pair of sense and antisense composite primers whose respective hybridization segments constitute, with respect to one another, a pair of sense and antisense primers for one of said target nucleotide sequences, each one of said target nucleotide sequences of the plurality targeted thus having a pair of sense and antisense composite primers which is intended for its amplification,
b) all the sense composite primers contain the same nucleotide tag and all the antisense composite primers contain in the same nucleotide, the tag of the sense composite primers being different from that of the antisense composite primers,
c) the sequence of the tag of the sense composite primers is absent from said nucleic acid or mixture of nucleic acids, or, at the very least, is only present therein at a frequency at least two times less than that predicted statistically for a random sequence of the same length, and the sequence of the tag of the antisense composite primers is absent from said nucleic acid or mixture of nucleic acids, or, at the very least, is only present therein at a frequency at least two times less than that predicted statistically for a random sequence of the same length,
d) the melting temperature of each composite primer (whether it is a sense or antisense primer) has a value 10 to 15° C. higher (limits inclusive) than that which its hybridization segment would exhibit when naked without tag,
e) each composite primer of said plurality of pairs has a sequence such that no composite primer of said plurality of pairs can form, with itself or with another composite primer of the same plurality, complete or partial base pairing for which the variation in free energy ΔG associated with the formation of this possible pairing would be greater than 14 kcal/mol, said variation in free energy ΔG being calculated using the “Primer Premier” software version 5.0 marketed by PREMIER Biosoft International,
and in that the thus selected plurality of antisens composite primer pairs is synthesized.

2. Plurality of pairs of sense and antisense composite primers specially adapted to the quantitative multiplex amplification of a plurality of target nucleotide sequences present in a nucleic acid or a mixture of nucleic acids, characterized in that it is obtainable using the method according to claim 1.

3. Plurality of pairs of sense and antisense composite primers according to claim 2, characterized in that said nucleic acid or mixture of nucleic acids is derived from mammalian cells.

4. Plurality of pairs of sense and antisense composite primers according to either one of claims 2 and 3, characterized in that said nucleic acid or mixture of nucleic acids is derived from human cells.

5. Plurality of pairs of sense and antisense composite primers according to any one of claims 2 to 4, characterized in that said nucleic acid or mixture of nucleic acids corresponds to the total genomic DNA of an organism or of a microorganism.

6. Plurality of pairs of sense and antisense composite primers according to any one of claims 2 to 4, characterized in that said nucleic acid or mixture of nucleic acids corresponds to complementary DNA produced in vitro from RNA of an organism or of a microorganism.

7. Plurality of pairs of sense and antisense composite primers according to any one of claims 2 to 6, characterized in that the tag of the sense composite primers, and also that of the antisense composite primers, each comprise, independently of one another, from 8 to 18 nucleotides, preferentially from 8 to 15 nucleotides, more preferentially 8 to 14 nucleotides, even more preferentially 9 to 12 nucleotides, very preferentially 10 nucleotides.

8. Plurality of pairs of sense and antisense composite primers according to any one of claims 2 to 7, characterized in that the sequence of the tag of the sense composite primers, and also that of the tag of the antisense composite primers, each consist of a chain of 10 nucleotides the GC content of which is between 20% and 60% (limits inclusive), preferentially between 20% and 50% (limits inclusive), very preferentially a GC content of 40%.

9. Plurality of pairs of sense and antisense composite primers according to any one of claims 2 to 8, characterized in that the tag of the sense composite primers and/or that of the antisense composite primers is/are selected from the group consisting of the sequence of SEQ ID NO:1, the sequence of SEQ ID NO:2, and the sequences complementary thereto, SEQ ID NO:47 and SEQ ID NO:48.

10. Plurality of pairs of sense and antisense composite primers according to any one of claims 2 to 9, characterized in that it comprises at least one pair of sense and antisense composite primers the respective sense and antisense hybridization segments of which have the following sequences:
the sequences of SEQ ID NO:3 and SEQ ID NO:4, or
the sequences of SEQ ID NO:7 and SEQ ID NO:8, or
the sequences of SEQ ID NO:9 and SEQ ID NO:10, or
the sequences of SEQ ID NO:11 and SEQ ID NO:12, or
the sequences of SEQ ID NO:13 and SEQ ID NO:14.

11. Plurality of pairs of sense and antisense composite primers according to any one of claims 2 to 9, characterized in that it comprises at least one pair of sense and antisense composite primers the respective sense and antisense hybridization segments of which have the following sequences:
SEQ ID NO:27 and SEQ ID NO:28, or
SEQ ID NO:29 and SEQ ID NO:30, or
SEQ ID NO:31 and SEQ ID NO:32, or
SEQ ID NO:33 and SEQ ID NO:34, or
SEQ ID NO:35 and SEQ ID NO:36.

12. Plurality of pairs of sense and antisense composite primers according to any one of claims 2 to 11, characterized in that the composite primers of said plurality of pairs each have a hybridization segment the melting temperature Tm of which is between 50 and 65° C., preferentially between 58 and 62° C., all limits inclusive.

13. Plurality of pairs of sense and antisense composite primers according to any one of claims 2 to 12, characterized in that the composite primers of said plurality of pairs each have a melting temperature Tm of greater than 65° C., preferentially of between 68° C. and 72° C., all limits inclusive.

14. Plurality of pairs of sense and antisense composite primers according to any one of claims 2 to 13, characterized in that said non-nucleotide component is a label for detecting nucleotide products.

15. Plurality of pairs of sense and antisense composite primers according to any one of claims 2 to 14, characterized in that it comprises from 2 to 15 pairs of sense and antisense composite primers.

16. Method for producing a plurality of pairs of sense and antisense composite primers according to any one of claims 2 to 15, characterized in that it comprises the following steps:
a) selected from:
pairs of sense and antisense hybridization segments which each form a pair of sense and antisense primers for one of said target nucleotide sequences, and
nucleotide tags which are absent from said nucleic acid or mixture of nucleic acids, or which at the very least are only present therein at a frequency at least two times less than that predicted statistically for a random sequence of the same length,
are a plurality of pairs of sense and antisense hybridization segments which covers the plurality of target nucleotide sequences targeted, and a pair of nucleotide tags,
the respective sequences of which are such that:
when one of the two selected tags is attached to the 5′ end of each selected sense hybridization segment, and the other of the two selected tags is attached to the 5′ end of each selected antisense hybridization segment, then:
each resulting sense or antisense composite primer has a melting temperature Tm with a value 10 to 15° C. greater (limits inclusive) than that which its hybridization segment would exhibit when naked without tag, and
each resulting sense or antisense composite primer has a sequence such that it cannot form, with itself or with another resulting composite primer, a complete or partial base pairing for which the variation in free energy ΔG associated with the formation of this pairing would be greater than 14 kcal/mol,
b) the plurality of pairs of sense and antisense composite primers which results from the selection of the plurality of pairs of hybridization segments and of the pair of tags made in step a), and of the addition of the sequence of one of the two selected tags to the 5′ end of each sense hybridization segment of the selected plurality, and of the addition of the sequence of the other of the two selected tags to the 5′ end of each antisense hybridization segment of the selected plurality, is produced.

17. Method for producing a plurality of pairs of composite primers according to any one of claims 2 to 15, characterized in that it comprises the following steps:
a) a plurality of pairs of sense and antisense hybridization segments is selected:
in which each pair of segments constitutes a pair of sense and antisense primers for each one of the target nucleotide sequences targeted, and
in which no segment can form, with itself or with another segment of this plurality, a complete or partial base pairing for which the variation in free energy ΔG associated with the formation of this is possible pairing would be greater than 14 kcal/mol, preferentially 13 kcal/mol, more preferentially 12 kcal/mol,
b) two nucleotide tags are selected:
the respective sequences of which are absent from said nucleic acid or mixture of nucleic acids, or at the very least which are only present therein at a frequency at least two times less than that predicted statistically for a random sequence of the same length, and
which have respective sequences such that their addition, for one, to the 5′ end of each one of the sense hybridization segments selected in step a) and, for the other, to the 5′ end of each one of the antisense hybridization segments selected in step a), does not produce a set of sense and antisense composite primers within which a composite primer would be capable of forming, with itself or with another composite primer of this set, a complete or partial base pairing, the formation of which would correspond to a variation in free energy ΔG of greater than 14 kcal/mol,
c) a plurality of pairs of sense and antisense composite primers is produced by adding the sequence of one of the two tags selected in step b) to the 5′ end of each sense hybridization segment selected in step a), and by adding the sequence of the other of the two tags selected in step b) to the 5′ end of each antisense hybridization segment selected in step a), whereby a plurality of pairs of composite primers according to any one of claims 2 to 15 is produced.

18. Method according to claim 17, characterized in that said hybridization segments, whether they are sense or antisense, each have (in the absence of a tag) a melting temperature Tm of between 50 and 65° C. (limits inclusive).

19. Plurality of pairs of composite primers according to any one of claims 2 to 15, characterized in that it can be obtained using a method according to any one of claims 16 to 18.

20. Pair of sense and antisense composite primers suitable for use within a plurality according to any one of claims 2 to 15, characterized in that it is selected from a plurality according to claim 19.

21. Method for producing a pair of tags suitable for use as a tag of sense composite primers and a tag of antisense composite primers in a plurality of pairs of sense and antisense composite primers according to any one of claims 2 to 15 and 19, which pair is termed universal, characterized in that it comprises the following steps:
a) at least 30 pairs of sense and antisense hybridization segments are selected:
which each form a pair of sense and antisense primers for a nucleotide target, so as to target at least 30 different nucleotide targets of aid nucleic acid or mixture of nucleic acids, and taking care that these at least 30 targets exhibit a uniform distribution throughout the length of said nucleic acid or mixture of nucleic acids, or at the very least in the region(s) in which are found the target nucleotide sequences whose amplification in multiplex is desired, and
each segment of which has a melting temperature Tm of between 50 and 65° C. (limits inclusive), thus constituting a set of pairs of test sense and antisense segments,
b) for each pair of test segments of the set, the maximum value of the variation in free energy ΔG that this pair can exhibit, by partial or complete base pairing of each one of the two segments with itself or with the other segment of the same pair, is determined,
c) two tags of different sequences are selected:
which are not present in said nucleic acid or mixture of nucleic acids, or at the very least which are only present therein at a frequency at least two times less than that predicted statistically for a random sequence of the same length, and
the addition of which, for one, to the 5′ end of each test sense segment and, for the other, to the 5′ end of each test antisense segment, leads to an increase of a value of between 10 and 15° C. (limits inclusive) in the melting temperature Tm of each one of the test segments, and
the addition of which, for one, to the 5′ end of each test sense segment and, for the other, to the 5′ end of each test antisense segment, does not for any of the pairs of test sense and antisense segments lead to an increase of more than 3 kcal/mol in said maximum value ΔG determined for each test pair in step b),
d) the two selected tags are produced.

22. Pair of nucleotide tags intended to cooperate with a plurality of sense and antisense hybridization segments so as to form a plurality of pairs of sense or antisense composite primers according to any one of claims 2 to 15 and 19, which pair is termed universal, characterized in that it is obtainable by the method according to claim 21.

23. Pair of nucleotide tags according to claim 22, characterized in that it is selected from the group of pairs of tags of respective sequences:
the sequences SEQ ID NO:1 and SEQ ID NO:2,
the sequence SEQ ID NO:1 and the sequence complementary to SEQ ID NO:2 (SEQ ID NO:48),
the sequence complementary to SEQ ID NO:1 (SEQ ID NO:47) and the sequence SEQ ID NO:2,
the sequence complementary to SEQ ID NO:1 (SEQ ID NO:47) and the sequence complementary to SEQ ID NO:2 (SEQ ID NO:48).

24. Nucleotide tag suitable for use as a tag in the sense composite primers or in the antisense composite primers of a plurality according to any one of claims 2 to 15 and 19, characterized in that it is selected from a pair of tags according to claim 22 or 23.

25. Nucleotide tag suitable for use as a tag in the sense composite primers or in the antisense composite primers of a plurality according to any one of claims 2 to 15 and 19, characterized in that its sequence:
consists of 10 nucleotides,
has a GC content of between 20% and 60% (limits inclusive), preferentially between 20% and 50% (limits inclusive), very preferentially a GC content of 40%, and
is absent from said nucleic acid or mixture of nucleic acids, or at the very most is only present therein at a frequency at least two times less than that statistically predicted for a random sequence of the same length.

26. Nucleotide tag according to claim 25, characterized in that, in addition, its sequence is such that the complete pairing with the chain of 10 nucleotides which constitutes the sequence completely complementary thereto exhibits a free energy of formation ΔG which does not exceed 11 kcal/mol.

27. Tag according to either one of claims 25 and 26, characterized in that its sequence is selected from the group consisting of the sequence of SEQ ID NO:1, the sequence of SEQ ID NO:2, and the sequences complementary thereto, SEQ ID NO:47 and SEQ ID NO:48.

28. Sense or antisense composite primer suitable for use within a plurality according to any one of claims 2 to 15, characterized in that it is selected from a pair of primers according to claim 20, and in that the tag which it contains is a tag according to any one of claims 24 to 27.

29. Method for amplifying in multiplex a plurality of target nucleotide sequences present in a nucleic acid or a mixture of nucleic acids, by hybridizations and elongations of a plurality of pairs of amplification primers, characterized in that said plurality of pairs of amplification primers is a plurality of pairs of sense and antisense composite primers according to any one of claims 2 to 15 and 19.

30. Method of amplification according to claim 29, characterized in that said plurality of target nucleotide sequences amplified in multiplex consists of 2 to 16 target sequences.

31. Method of amplification according to claim 29 or 30, characterized in that said target nucleotide sequences consist of 90 to 500 bp, preferentially of 90 to 300 bp.

32. Method for the quantitative multiplex amplification of a plurality of target nucleotide sequences present in a nucleic acid or a mixture of nucleic acids, by hybridizations and elongations of a plurality of pairs of amplification primers, characterized in that the method according to any one of claims 29 to 31 is used, carrying out said hybridizations and/or elongations in the presence of an agent which facilitates DNA strand separation, such as dimethyl sulphoxide (DMSO) or triethylammonium acetate (TEAA).

33. Method of amplification according to claim 32, characterized in that said hybridizations and elongations are carried out with successive hybridization-elongation-denaturation cycles until the amplifications of said target nucleotide sequences have exponential phase kinetics.

34. Method of amplification according to either one of claims 32 and 33, characterized in that all the pairs of composite primers are used in equimolar concentration.

35. Method for determining the presence or absence of at least one genomic rearrangement within a genetic material B relative to a reference genetic material A, characterized in that:
at least one nucleotide target which constitutes a marker for the rearrangement(s) sought is selected, and in that
the method of amplification according to any one of claims 29 to 31, or else that according to any one of claims 32 to 34, is applied to said genetic material B, using, for each target selected, a pair of composite primers which is selected from plurality of pairs of composite primers according to any one of claims 2 to 15 and 19, and which is suitable for the amplification of this target from the genetic material B,
said material B being considered as exhibiting said genetic rearrangement when the result of amplification of said at least one marker target, obtained from the material B, is significantly different from that which is obtained from the reference material A under identical conditions, and
said material B being considered as not exhibiting said genetic rearrangement when the result of amplification of said at least one marker target, obtained from the material B, is not significantly different from that which is obtained from the reference material A under identical conditions.

36. Method according to claim 35, characterized in that said at least one genomic rearrangement is a gene rearrangement.

37. Method according to claim 35, characterized in that said at least one genomic rearrangement is a chromosomal rearrangement.

38. Method according to any one of claims 35 to 37, characterized in that said genetic material B comprises at least one human gene.

39. Method for determining at least one of the limits of one or more genomic rearrangement(s) which has (have) been detected within a genetic material B by comparison with a reference genetic material A, characterized in that:
a) a candidate region within which said at least one limit is potentially located is selected,
b) for each rearrangement, a set of nucleotide targets is chosen, among which at least one is chosen to constitute marker for this rearrangement, the other target(s) being chosen on both sides or on one or other sides of this marker target inside the candidate region chosen in step a) so as to cover the extent of this candidate region,
c) the method according to any one of claims 29 to 31, or else the method according to any one of claims 32 to 34, is applied to said genetic material B, using, for each target of said chosen set, at least one pair of composite primers which is chosen from a plurality of pairs of composite primers according to any one of claims 2 to 15 and 19, and which is suitable for amplifying this target from said genetic material A,
d) for each target, the intensity of amplification thus obtained from said genetic material B is measured, and it is compared to the control intensity which is obtained for this same target under the same conditions but by applying said method of amplification to said reference genetic material A,
e) it is determined whether, within the chosen set of targets, at least one target is amplified with an intensity not significantly different from the control intensity, and, if this is not the case, steps a) to e) are repeated while broadening the candidate region chosen in step a),
said at least one limit of the or of each one of the rearrangements within said genetic material B being considered to be within a zone between:
the position of the marker target for said rearrangement, and
the position of the target which has been amplified with an intensity not significantly different from the control intensity or, if there are several of them, with that which is closest to said marker target,
f) if desired, the precision of determination of said limit is refined, by gradually walking into the zone determined in step e) above, by repeating steps a) to e), choosing as candidate region in step a) the zone identified in the immediately preceding step e), and choosing in step b) a set of nucleotide targets which covers this zone identified in step e).

40. Method for producing a genomic rearrangement map, characterized in that the limits of at least one genomic rearrangement are determined using the method according to claim 39, and in that these limits are recorded on a gene or chromosome map.

41. Method for identifying, and optionally isolating, at least one gene liable to be involved in a genetic disease, characterized in that:
the method for determining the presence or absence of at least one genomic rearrangement according to any one of claims 35 to 38 is carried out on a genetic material B derived from organisms exhibiting said genetic disease, a genomic material which is comparable but derived from control organisms serving as reference genomic material A, so as to detect the rearrangement(s) present in the material B relative to the material A, and in that
the gene(s) affected by the detected rearrangement(s) is (are) identified, and optionally isolated,
this (these) identified and optionally isolated gene(s) corresponding to the gene(s) liable to be involved in said genetic disease.

42. Method for diagnosing a genetic disease from which an individual might suffer, or for estimating a propensity for this individual to develop such a disease, characterized in that the method for determining the presence or absence of at least one genomic rearrangement according to any one of claims 35 to 38 is carried out on a sample of genetic material representative of said genetic disease, and in that said diagnosis is considered to be positive, or, where appropriate, said propensity is considered to be high, when said at least one genetic rearrangement is determined as being present in said sample and, conversely,
said diagnosis is considered to be negative, or, where appropriate, said propensity is considered to be low, when said at least one genomic rearrangement is determined as being absent from said sample.

43. Kit for carrying out a method of amplification according to any one of claims 29 to 31, and/or a method of amplification according to any one of claims 32 to 34, and/or a method for determining the presence or absence of at least one genomic rearrangement according to any one of claims 35 to 38, and/or a method for determining the limits of one or more genetic rearrangement(s) according to claim 39, and/or a method for identifying at least one gene involved in a genetic disease according to claim 41, and/or a diagnostic or prognostic method according to claim 42, characterized in that it comprises at least one pair of composite primers according to claim 20, and/or at least one composite primer according to claim 28, and/or at least one tag according to any one of claims 24 to 27, optionally combined with an amplification primer and/or with a label for detecting nucleotide products.


SEQUENCE LISTING (48 sequences)

SEQ ID NO: 1 - DNA, 10 bp, artificial sequence
  non-hybridizing primer tag
cgttagatag

SEQ ID NO: 2 - DNA, 10 bp, artificial sequence
  non-hybridizing primer tag
gatagggtta

SEQ ID NO: 3 - DNA, 20 bp, artificial sequence
  hybridization segment
actccatctccttgtgctct

SEQ ID NO: 4 - DNA, 20 bp, artificial sequence
  hybridization segment
cgctattcaacaagctcatg

SEQ ID NO: 5 - DNA, 20 bp, artificial sequence
  hybridization segment
ggtaaaacacattcctttgg

SEQ ID NO: 6 - DNA, 20 bp, artificial sequence
  hybridization segment
atatgtgagcttccattggt

SEQ ID NO: 7 - DNA, 20 bp, artificial sequence
  hybridization segment
atgtttaacaaccgccagca

SEQ ID NO: 8 - DNA, 22 bp, artificial sequence
  hybridization segment
tcttcctttcagatgatgcaga

SEQ ID NO: 9 - DNA, 21 bp, artificial sequence
  hybridization segment
gacatggtgctgtgtgtgagc

SEQ ID NO: 10 - DNA, 21 bp, artificial sequence
  hybridization segment
tccgcctttagaagtccaagt

SEQ ID NO: 11 - DNA, 20 bp, artificial sequence
  hybridization segment
tgaagctgtgtggctgaaac

SEQ ID NO: 12 - DNA, 20 bp, artificial sequence
  hybridization segment
tagccagggtgtctcaaaga

SEQ ID NO: 13 - DNA, 20 bp, artificial sequence
  hybridization segment
taccagtcatcgggcagaac

SEQ ID NO: 14 - DNA, 21 bp, artificial sequence
  hybridization segment
aatgtcagaggcaggacacag

SEQ ID NO: 15 - DNA, 30 bp, artificial sequence
  composite primer
cgttagatagactccatctccttgtgctct

SEQ ID NO: 16 - DNA, 30 bp, artificial sequence
  composite primer
gatagggttacgctattcaacaagctcatg

SEQ ID NO: 17 - DNA, 30 bp, artificial sequence
  composite primer
cgttagatagggtaaaacacattcctttgg

SEQ ID NO: 18 - DNA, 30 bp, artificial sequence
  composite primer
gatagggttaatatgtgagcttccattggt

SEQ ID NO: 19 - DNA, 30 bp, artificial sequence
  composite primer
cgttagatagatgtttaacaaccgccagca

SEQ ID NO: 20 - DNA, 32 bp, artificial sequence
  composite primer
gatagggttatcttcctttcagatgatgcaga

SEQ ID NO: 21 - DNA, 31 bp, artificial sequence
  composite primer
cgttagataggacatggtgctgtgtgtgagc

SEQ ID NO: 22 - DNA, 31 bp, artificial sequence
  composite primer
gatagggttatccgcctttagaagtccaagt

SEQ ID NO: 23 - DNA, 30 bp, artificial sequence
  composite primer
cgttagatagtgaagctgtgtggctgaaac

SEQ ID NO: 24 - DNA, 30 bp, artificial sequence
  composite primer
gatagggttatagccagggtgtctcaaaga

SEQ ID NO: 25 - DNA, 30 bp, artificial sequence
  composite primer
cgttagatagtaccagtcatcgggcagaac

SEQ ID NO: 26 - DNA, 31 bp, artificial sequence
  composite primer
gatagggttaaatgtcagaggcaggacacag

SEQ ID NO: 27 - DNA, 17 bp, artificial sequence
  hybridization segment
ccctggtgcgatggggt

SEQ ID NO: 28 - DNA, 19 bp, artificial sequence
  hybridization segment
ggcacggcgggacaagtag

SEQ ID NO: 29 - DNA, 19 bp, artificial sequence
  hybridization segment
agtcgtgctgtcctgaacg

SEQ ID NO: 30 - DNA, 22 bp, artificial sequence
  hybridization segment
tcttcttccttcttttcttcaa

SEQ ID NO: 31 - DNA, 23 bp, artificial sequence
  hybridization segment
gcatcctcctactcttctcctgg

SEQ ID NO: 32 - DNA, 20 bp, artificial sequence
  hybridization segment
agcctccctcaaataggtct

SEQ ID NO: 33 - DNA, 18 bp, artificial sequence
  hybridization segment
tggggctaggaggtccct

SEQ ID NO: 34 - DNA, 24 bp, artificial sequence
  hybridization segment
cctcccctttatgagactatccta

SEQ ID NO: 35 - DNA, 19 bp, artificial sequence
  hybridization segment
agaggcagggaatgaagaa

SEQ ID NO: 36 - DNA, 20 bp, artificial sequence
  hybridization segment
gggtcaccttgatattcaca

SEQ ID NO: 37 - DNA, 27 bp, artificial sequence
  composite primer
cgttagatagccctggtgcgatggggt

SEQ ID NO: 38 - DNA, 29 bp, artificial sequence
  composite primer
gatagggttaggcacggcgggacaagtag

SEQ ID NO: 39 - DNA, 29 bp, artificial sequence
  composite primer
cgttagatagagtcgtgctgtcctgaacg

SEQ ID NO: 40 - DNA, 32 bp, artificial sequence
  composite primer
gatagggttatcttcttccttcttttcttcaa

SEQ ID NO: 41 - DNA, 33 bp, artificial sequence
  composite primer
cgttagataggcatcctcctactcttctcctgg

SEQ ID NO: 42 - DNA, 30 bp, artificial sequence
  composite primer
gatagggttaagcctccctcaaataggtct

SEQ ID NO: 43 - DNA, 28 bp, artificial sequence
  composite primer
cgttagatagtggggctaggaggtccct

SEQ ID NO: 44 - DNA, 34 bp, artificial sequence
  composite primer
gatagggttacctcccctttatgagactatccta

SEQ ID NO: 45 - DNA, 29 bp, artificial sequence
  composite primer
cgttagatagagaggcagggaatgaagaa

SEQ ID NO: 46 - DNA, 30 bp, artificial sequence
  composite primer
gatagggttagggtcaccttgatattcaca

SEQ ID NO: 47 - DNA, 10 bp, artificial sequence
  non-hybridizing primer tag
ctatctaacg

SEQ ID NO: 48 - DNA, 10 bp, artificial sequence
  non-hybridizing primer tag
taaccctatc